(19) United States
(12) Patent Application Publication
(10) Pub. No.: US 2009/0203706 A1
(43) Pub. Date: Aug. 13, 2009

(54) LYSINE-BASED POLYMERIC LINKERS

(75) Inventors: Hong Zhao, Edison, NJ (US); Prasanna Reddy, Somerset, NJ (US)

Correspondence Address: LUCAS & MERCANTI, LLP, 475 PARK AVENUE SOUTH, 15TH FLOOR, NEW YORK, NY, 10016, US

(73) Assignee: ENZON PHARMACEUTICALS, INC., Bridgewater, NJ (US)

(21) Appl. No.: 12/402,980

(22) Filed: Mar. 12, 2009

Related U.S. Application Data
(63) Continuation of application No. PCT/US2007/078594, filed on Sep. 15, 2007.
(60) Provisional application No. 60/844,945, filed on Sep. 15, 2006.
(60) Provisional application No. 60/861,349, filed on Nov. 27, 2006.
(60) Provisional application No. 60/911,734, filed on Apr. 13, 2007.

(51) Int. Cl. A61K 31/197; C07C 229/04; C07D 207/40; C07D 241/04; A61K 31/4025; A61K 31/496

(52) U.S. Cl. 514/252.11; 562/564; 548/546; 544/357; 514/563; 514/422

(57) ABSTRACT

The present invention provides polymeric linkers containing branching moieties. Methods of making the polymeric linkers and methods of making conjugates using the same are also disclosed.

### CROSS-REFERENCE TO RELATED APPLICATION

This patent application is a continuation of PCT/US2007/078594, filed Sep. 15, 2007, which claims the benefit of priority from U.S. Provisional Patent Application Ser. Nos. 60/844,945 filed Sep. 15, 2006, 60/861,349 filed Nov. 27, 2006 and 60/911,734 filed Apr. 13, 2007, the contents of each of which are incorporated herein by reference.

### FIELD OF THE INVENTION

The present invention relates to drug delivery systems. In particular, the invention relates to polymer-based drug delivery systems containing a branching moiety providing multiple terminal amine groups which improve loading and delivery of certain biologically active moieties.

### BACKGROUND OF THE INVENTION

Over the years, numerous methods have been proposed for delivering therapeutic agents into the body and improving bioavailability of those medicinal agents. One of the attempts is to include such medicinal agents as part of a soluble transport system. Such transport systems can include permanent conjugate-based systems or prodrugs. In particular, polymeric transport systems can improve the solubility and stability of medicinal agents. For example, the conjugation of water-soluble polyalkylene oxides with therapeutic moieties such as proteins and polypeptides is known. See, for example, U.S. Pat. No. 4,179,337, the disclosure of which is incorporated herein by reference The '337 patent discloses that physiologically active polypeptides modified with PEG circulate for extended periods in vivo, and have reduced immunogenicity and antigenicity.

Additional improvements have been also realized. For example, polymer-based drug delivery platform systems containing benzyl elimination systems, trialkyl lock systems, etc. were disclosed by Enzon Pharmaceuticals as a means of releasably delivering proteins, peptides and small molecules. See also Greenwald, et al. J. Med. Chem. Vol. 42, No. 18, 3657-3667; Greenwald, et al. J. Med. Chem. Vol. 47, No. 3, 726-734; Greenwald, et al. J. Med. Chem. Vol. 43, No. 3, 475-487. The contents of each of the foregoing are hereby incorporated herein by reference.

To conjugate therapeutic agents such as small molecules and oligonucleotides to polyalkylene oxides, the hydroxyl end-groups of the polymer must first be converted into reactive functional groups. This process is frequently referred to as “activation” and the product is called an “activated polyalkylene oxide”. Other polymers are similarly activated.

In spite of the attempts and advances, further improvements in PEG and polymer conjugation technology such as polymers with higher loading of therapeutic agents have therefore been sought. The present invention addresses this need and others.

### SUMMARY OF THE INVENTION

In order to overcome the above problems and improve the technology for drug delivery, there are provided new branched polymers and conjugates made therewith.

In one aspect of the invention, there are provided compounds of Formula (I):

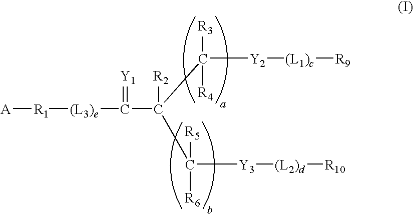

wherein:

R₁ is a substantially non-antigenic water-soluble polymer;

A is a capping group or

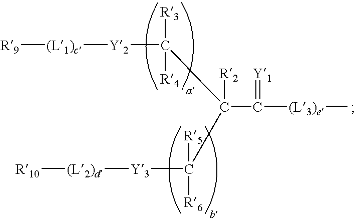

L₁₋₃ and L′₁₋₃ are independently selected bifunctional linkers;

Y₁ and Y′₁ are independently O, S, or NR₂₀;

R₂₋₇, R′₂₋₆, and R₂₀ are independently selected from among hydrogen, C₁₋₆ alkyl, C₂₋₆ alkeny, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, and substituted arylcarbonyloxy;

R₉₋₁₀ and R′₉₋₁₀ are independently selected from among hydrogen, OH, leaving groups, functional groups, targeting groups, diagnostic agents and biologically active moieties;

(a) and (a′) are independently zero or a positive integer;

(b) and (b′) are independently a positive integer; and

(c), (c′), (d), (d′), (e) and (e′) are independently zero or 1.

In certain preferred aspects of the invention, the polymeric drug-delivery systems include lysine.

In some preferred aspects, at least one functional group attached to the branching moiety of the invention is conjugated to a targeting moiety.

In some preferred aspects, at least one functional group attached to the branching moiety of the invention is conjugated to a biologically active moiety.

In some particularly preferred aspects, R₁ includes a linear or branched poly(ethylene glycol) residue with molecular weight of from about 5,000 to about 60,000, Y₁ and Y′₁ are O, Y₂₋₃ and Y′₂₋₃ are NH, (a) and (a′) are zero or one, (b) and (b′) are from about 2 to about 4, (c), (c′), (d), and (d′) are zero, and (e) and (e′) are 1. In one particular aspect R₂₋₇, R′₂₋₆ and R₂₀ are selected from among hydrogen, methyl and ethyl, and each is more preferably hydrogen.

In another aspect of the invention, there are provided methods of preparing the compounds described herein and methods of treatment using the compounds described herein.

One advantage of the branching moiety containing polymeric transport systems described herein is that the artisans are able to increase the loadings of medicinal agents. A further advantage of the polymeric systems described herein allows attaching a second agent. Multiple substitutions on the branching moiety will provide the artisans in the art to be able to attach a second drug to have synergistic effect for therapy or a targeting group for selectively targeted delivery. The polymeric delivery systems described herein allow targeting medicinal agents into the site of treatment.

Another advantage of the branching moiety-based polymeric transport systems described herein is that the polymeric delivery systems have improved stability. Without being bound by any theories, hydrophobic microenvironment around the covalent linkage between polymers and a moiety such functional groups, biologically active moieties and targeting groups inhibits the covalent linkage from exposing to basic aqueous medium or enzymes, which can modify the covalent linkage, and thereby stabilizes the covalent linkage. The stability of the polymeric systems also allows long-term storage prior to attaching to targeting groups or biologically active moieties.

For purposes of the present invention, the terms “a biologically active moiety” and “a residue of a biologically active moiety” shall be understood to mean that portion of a biologically active compound which remains after the biologically active compound has undergone a substitution reaction in which the transport carrier portion has been attached.

Unless otherwise defined, for purposes of the present invention:

the term “alkyl” shall be understood to include straight, branched, substituted, e.g. halo-, alkoxy-, and nitro-C₁₋₁₂ alkyls, C₃₋₈ cycloalkyls or substituted cycloalkyls, etc.;

the term “substituted” shall be understood to include adding or replacing one or more atoms contained within a functional group or compound with one or more different atoms;

the term “substituted alkyls” include carboxyalkyls, aminoalkyls, dialkylaminos, hydroxyalkyls and mereaptoalkyls;

the term “substituted cycloalkyls” include moieties such as 4-chlorocyclohexyl; aryls include moieties such as napthyl; substituted aryls include moieties such as 3-bromophenyl; aralkyls include moieties such as toluoyl; heteroalkyls include moieties such as ethylthiophene;

the term “substituted heteroalkyls” include moieties such as 3-methoxy-thiophene; alkoxy includes moieties such as methoxy; and phenoxy includes moieties such as 3-nitrophenoxy;

the term “halo” shall be understood to include fluoro, chloro, iodo and bromo; and

the terms “sufficient amounts” and “effective amounts” for purposes of the present invention shall mean an amount which achieves a therapeutic effect as such effect is understood by those of ordinary skill in the art.

### BRIEF DESCRIPTION OF THE DRAWINGS

FIG. 1 schematically illustrates methods of synthesis described in Examples 1-2.

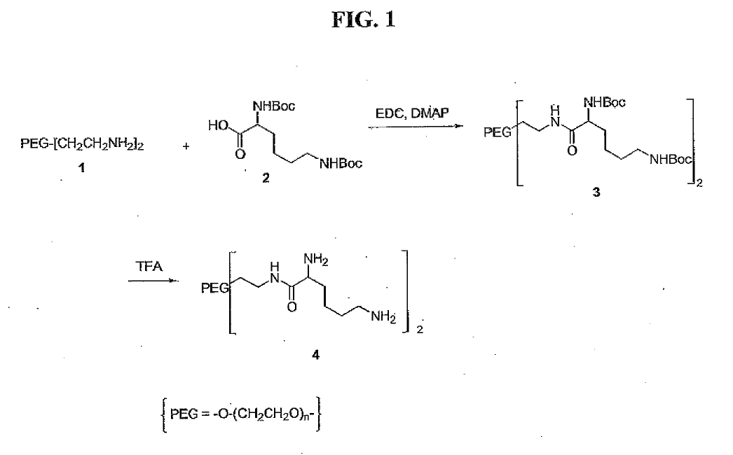

FIG. 2 schematically illustrates methods of synthesis described in Examples 3-8.

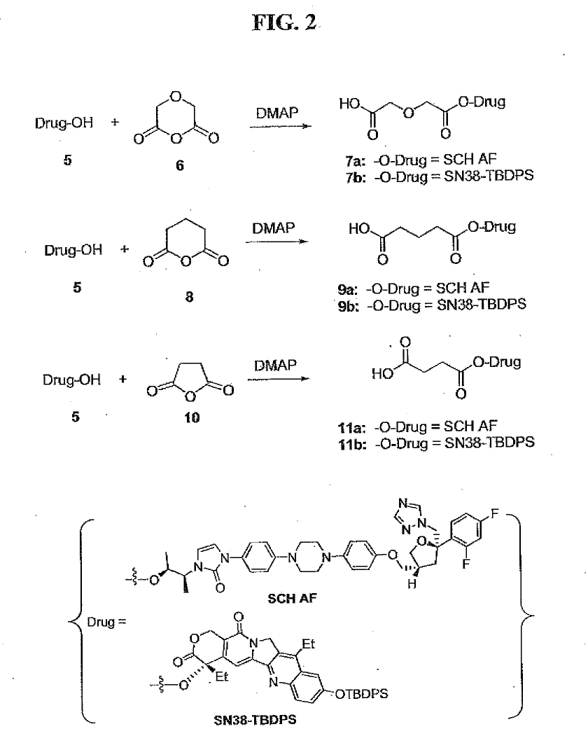

FIG. 3 schematically illustrates methods of synthesis described in Examples 9-14.

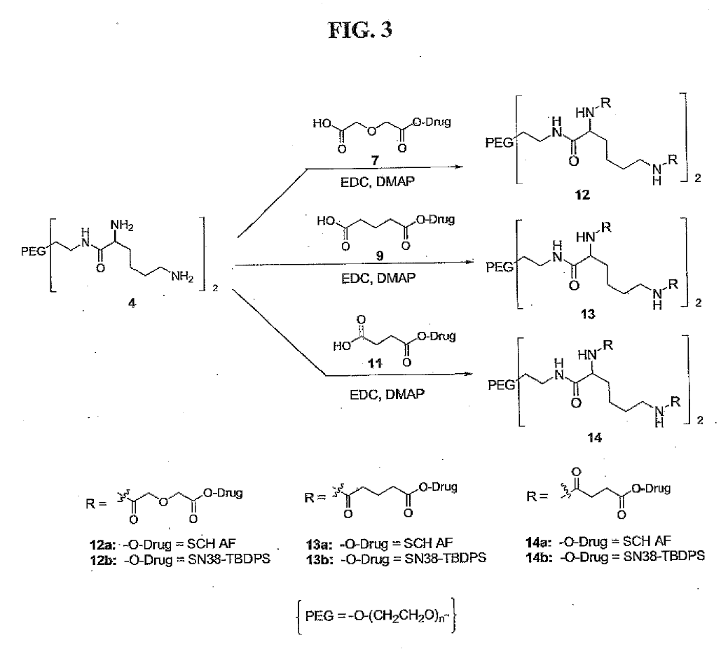

FIG. 4 schematically illustrates methods of synthesis described in Examples 15-17.

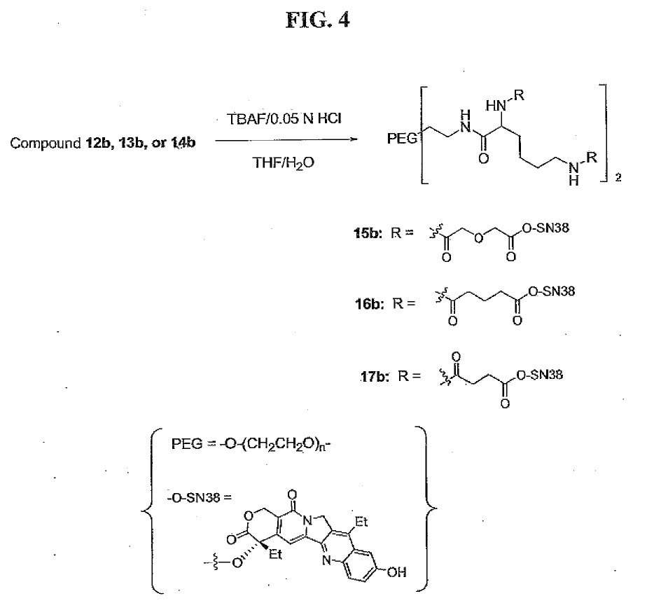

FIG. 5 schematically illustrates methods of synthesis described in Examples 18-21.

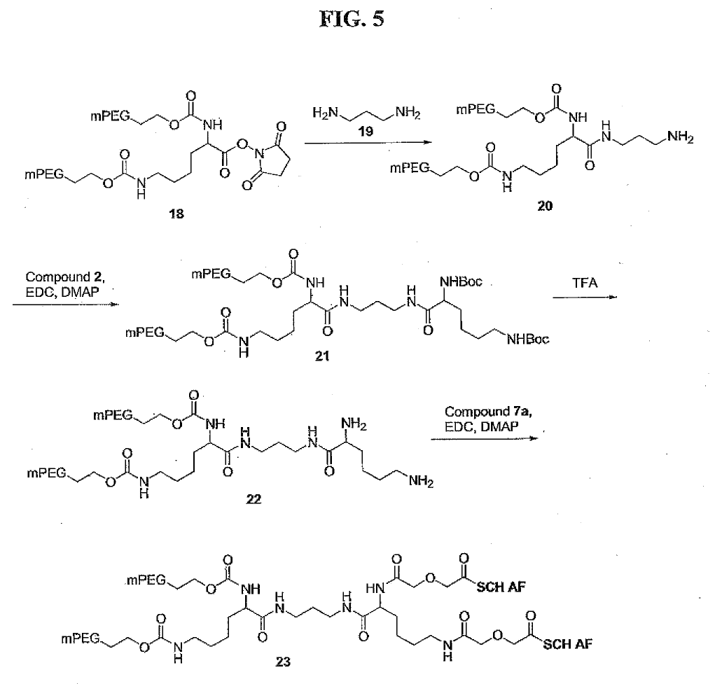

### DETAILED DESCRIPTION OF THE INVENTION

### A. Overview

In one aspect of the present invention, there are provided compounds of Formula (I):

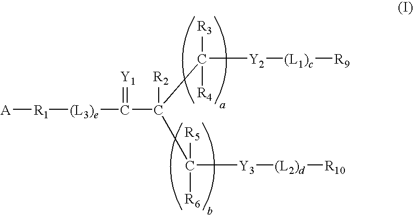

wherein:

R₁ is a substantially non-antigenic water-soluble polymer;

A is a capping group or

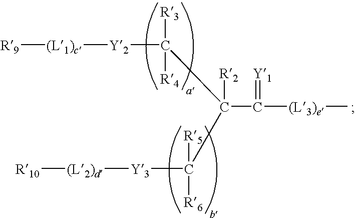

L₁₋₃ and L′₁₋₃ are independently selected bifunctional linkers;

Y₁ and Y′₁ are independently O, S, or NR₂₀;

Y₂₋₃ and Y′₂₋₃ are independently O, S, SO, SO₂ or NR₇;

R₂₋₇, R′₂₋₆, and R₂₀ are independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ akanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, and substituted arylcarbonyloxy;

R₉₋₁₀ and R′₉₋₁₀ are independently selected from among hydrogen, OH, leaving groups, functional groups, targeting groups, diagnostic agents and biologically active moieties;

(a) and (a′) are independently zero or a positive integer, preferably zero or an integer from 1 to 3 and more preferably zero;

(b) and (b′) are independently a positive integer, preferably from about 1 to about 10, more preferably about 2 to about 6 and most preferably 4; and

(c), (c′), (d), (d′), (e) and (e′) are independently zero or 1.

Within those aspects of the invention, the substituents contemplated for substitution, where the moieties corresponding to R₂₋₇, R′₂₋₆, and R₂₀ are indicated as being possibly substituted can include, for example, acyl, amino, amido, amidine, ara-alkyl, aryl, azido, alkylmercapto, arylmercapto, carbonyl, carboxylate, cyano, ester, ether, formyl, halogen, heteroaryl, heterocycloalkyl, hydroxy, imino, nitro, thiocarbonyl, thioester, thioacetate, thioformate, alkoxy, phosphoryl, phosphonate, phosphinate, silyl, sulfhydryl, sulfate, sulfonate, sulfamoyl, sulfonamide, and sulfonyl.

In another aspect of the invention, the biological moieties include —NH₂ containing moieties, —OH containing moieties and —SH containing moieties.

In yet another aspect, A can be selected from among H, NH₂, OH, CO₂H, C₁₋₆ alkoxy, and C₁₋₆ alkyls. In some other preferred embodiments, A can be methyl, ethyl, methoxy, ethoxy, H, and OH. A is more preferably methyl or methoxy.

In one particular embodiment, compounds described herein have the formula (II):

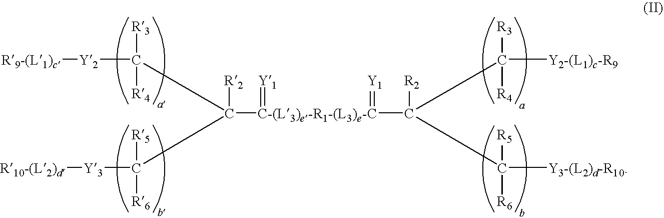

In some preferred embodiments, compounds described herein can be, for example,

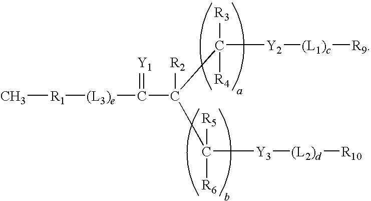

In more preferred embodiments, compounds described herein can be, for example,

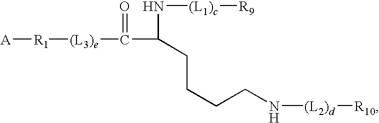

wherein, A is a capping group or

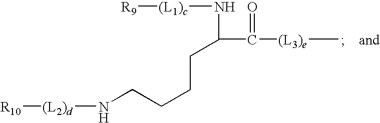

all other variables are as previously defined.

In some preferred embodiments, R₂₋₇, R′₂₋₆, and R₂₀ are independently hydrogen or CH₃. In some particularly preferred embodiments, R₂₋₈, R′₂₋₈, and R₂₀ are all hydrogen or CH₃. In other particular embodiments, R₃₋₆ and R′₃₋₆ include hydrogen and CH₃. In yet other particular embodiments, Y₁ includes O and NR₂₀, and R₂₋₈, R′₂₋₈, and R₄ includes hydrogen, C₁₋₆ alkyls, cycloalkyls, aryls, and aralkyl groups.

### B. Substantially Non-Antigenic Water-Soluble Polymers

Polymers employed in the compounds described herein are preferably water soluble polymers and substantially non-antigenic such as polyalkylene oxides (PAO's).

In one aspect of the invention, the compounds described herein include a linear, terminally branched or multi-armed polyalkylene oxide. In some preferred embodiments of the invention, the polyalkylene oxide includes polyethylene glycol and polypropylene glycol.

The polyalkylene oxide has an average molecular weight from about 2,000 to about 100,000 daltons, preferably from about 5,000 to about 60,000 daltons. The polyalkylene oxide can be more preferably from about 5,000 to about 25,000 or alternatively from about 20,000 to about 45,000 daltons. In some particularly preferred embodiments, the compounds described herein include the polyalkylene oxide having an average molecular weight of from about 12,000 to about 20,000 daltons or from about 30,000 to about 45,000 daltons. In one particular embodiment, polymeric portion has a molecular weight of about 12,000 or 40,000 daltons.

The polyalkylene oxide includes polyethylene glycols and polypropylene glycols. More preferably, the polyalkylene oxide includes polyethylene glycol (PEG). PEG is generally represented by the structure:

—O—(CH₂CH₂O)ₙ—

where (n) represents the degree of polymerization for the polymer, and is dependent on the molecular weight of the polymer. Alternatively, the polyethylene glycol (PEG) residue portion of the invention can be selected from among:

—Y₇₁—(CH₂CH₂O)ₙ—CH₂CH₂Y₇₁—,

—Y₇₁—(CH₂CH₂O)ₙ—CH₂C(═Y₇₂)—Y₇₁—,

—Y₇₁—C(═Y₇₂)—(CH₂)ₐ₇₁—Y₇₃—(CH₂CH₂O)ₙ—CH₂CH₂—Y₇₃—(CH₂)ₐ₇₁—C(═Y₇₂)—Y₇₁—, and

—Y₇₁—(CR₇₁R₇₂)ₐ₇₂—Y₇₃—(CH₂)_{b71}—O—(CH₂CH₂O)ₙ—(CH₂)_{b71}—Y₇₃—(CR₇₁R₇₂)ₐ₇₂—Y₇₁,

wherein:

Y₇₁ and Y₇₃ are independently O, S, SO, SO₂, NR₇₃ or a bond;

Y₇₂ is O, S, or NR₇₄;

R₇₁₋₇₄ are independently the same moieties which can be used for R₂;

(a71), (a72), and (b71) are independently zero or a positive integer, preferably 0-6, and more preferably 1; and

(n) is an integer from about 10 to about 2300.

Branched or U-PEG derivatives are described in U.S. Pat. Nos. 5,643,575, 5,919,455, 6,113,906 and 6,566,506, the disclosure of each of which is incorporated herein by reference. A non-limiting list of such polymers corresponds to polymer systems (i)-(vii) with the following structures:

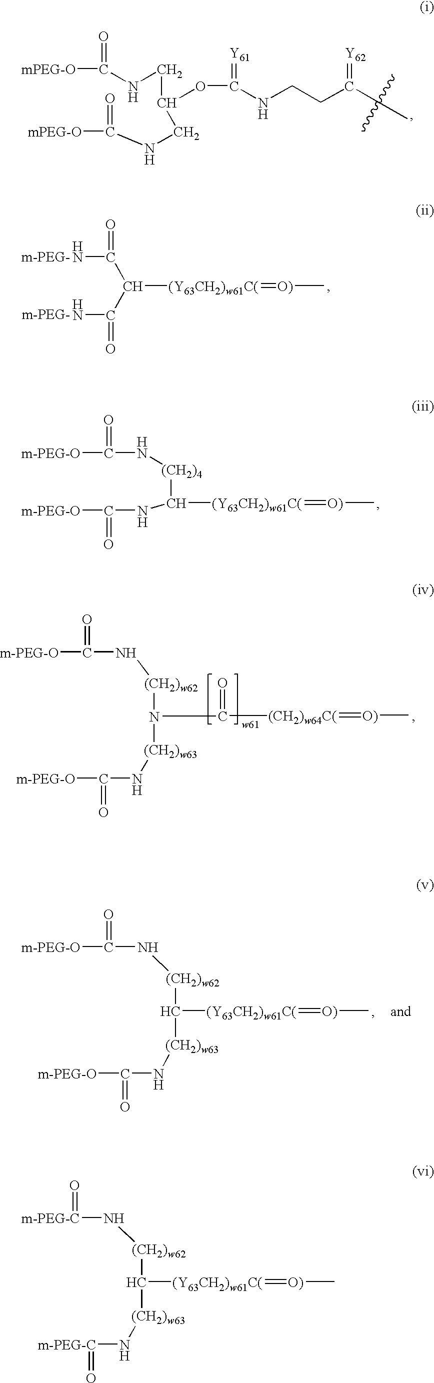

wherein:

Y₆₁₋₆₂ are independently O, S or NR₆₁;

Y₆₃ is O, NR₆₂, S, SO or SO₂

(w62), (w63) and (w64) are independently 0 or a positive integer;

(w61) is 0 or 1;

mPEG is methoxy PEG
wherein PEG is previously defined and a total molecular weight of the polymer portion is from about 2,000 to about 100,000 daltons; and

R₆₁ and R₆₂ are independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, and substituted and arylcarbonyloxy.

In yet another aspect, the polymers include multi-arm PEG-OH or “star-PEG” products such as those described in NOF Corp. Drug Delivery System catalog, Ver. 8, April 2006, the disclosure of which is incorporated herein by reference. The polymers can be converted into suitably activated forms, using the activation techniques described in U.S. Pat. Nos. 5,122,614 or 5,808,096 patents. Specifically, such PEG can be of the formula:

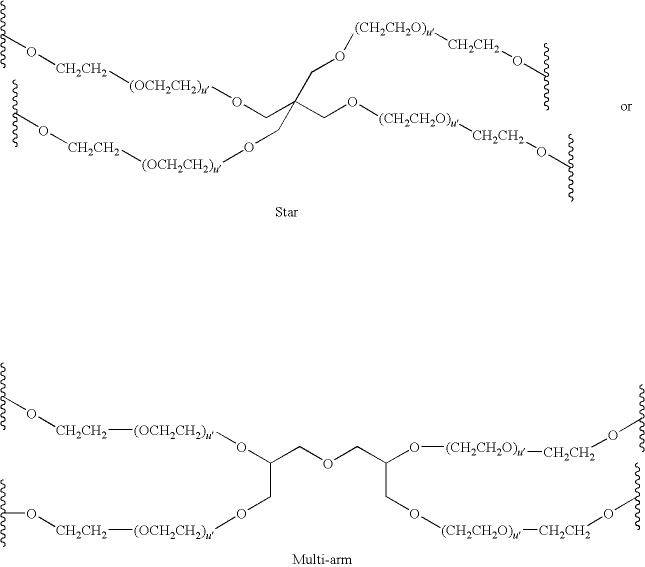

wherein;

(u′) is an integer from about 4 to about 455; and up to 3 terminal portions of the residue is/are capped with a methyl or other lower alkyl.

In some preferred embodiments, all 4 of the PEG arms can be converted to suitable activating groups, for facilitating attachment to aromatic groups. Such compounds prior to conversion include:

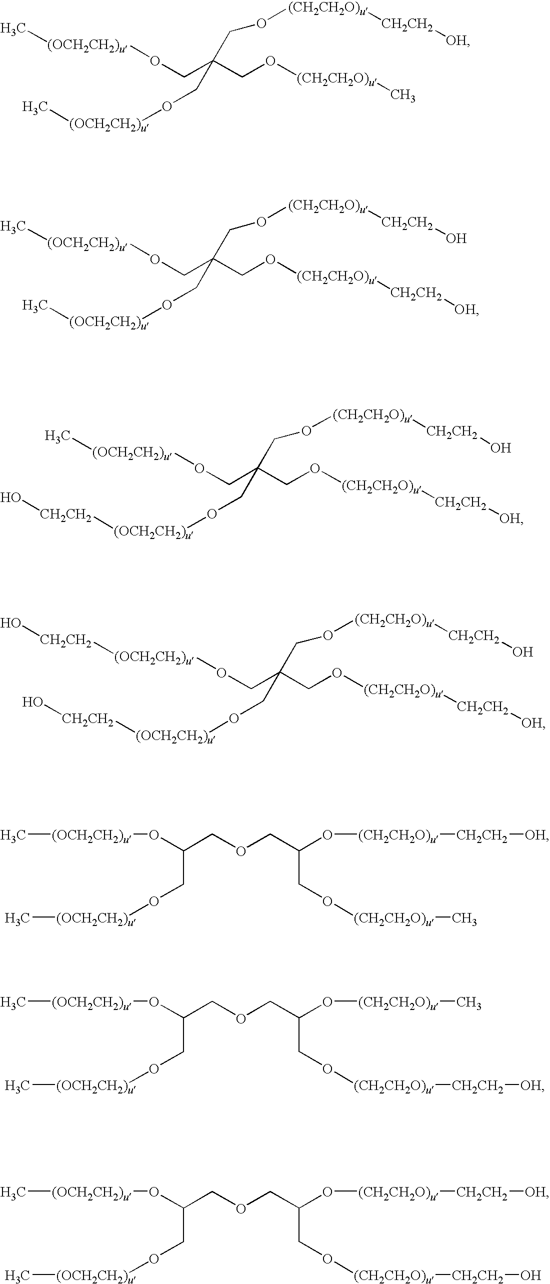

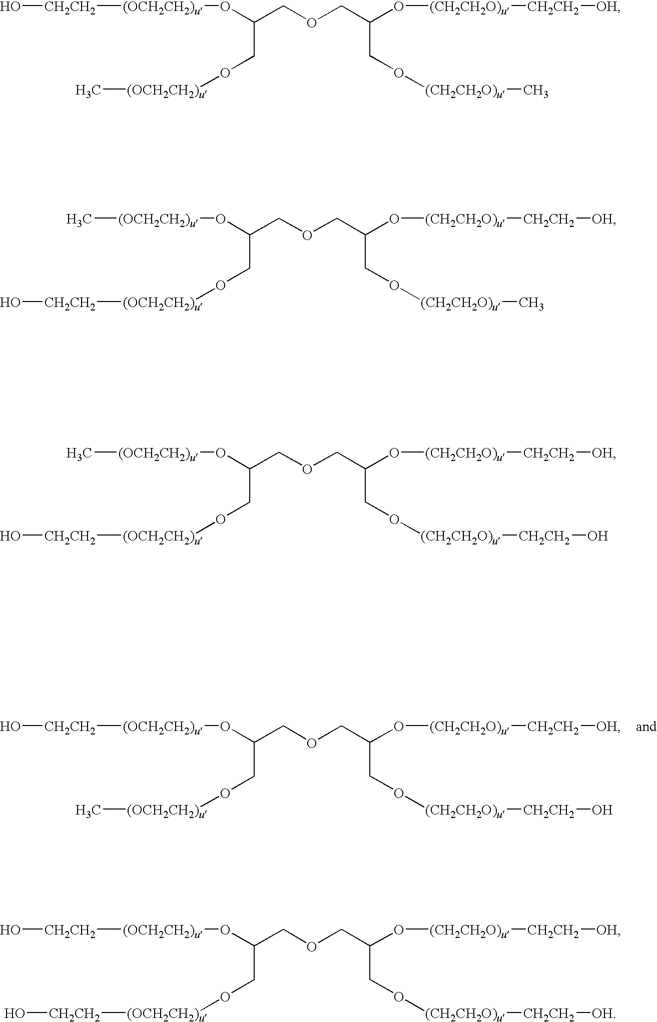

The polymeric substances included herein are preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof provided that the water solubility of the block copolymers is maintained.

In a further embodiment and as an alternative to PAO-based polymers, one or more effectively non-antigenic materials such as dextran, polyvinyl alcohols, carbohydrate-based polymers, hydroxypropylmethacrylamide (HPMA), polyalkylene oxides, and/or copolymers thereof can be used. See also commonly-assigned U.S. Pat. No. 6,153,655, the contents of which are incorporated herein by reference. It will be understood by those of ordinary skill that the same type of activation is employed as described herein as for PAO's such as PEG. Those of ordinary skill in the art will further realize that the foregoing list is merely illustrative and that all polymeric materials having the qualities described herein are contemplated. For purposes of the present invention, “substantially or effectively non-antigenic” means all materials understood in the art as being nontoxic and not eliciting an appreciable immunogenic response in mammals.

In some aspects, polymers having terminal amine groups can be employed to make the compounds described herein. The methods of preparing polymers containing terminal amines in high purity are described in U.S. patent application Ser. Nos. 11/508,507 and 11/537,172, the contents of each of which are incorporated by reference. For example, polymers having azides react with phosphine-based reducing agent such as triphenylphosphine or an alkali metal borohydride reducing agent such as NaBH₄. Alternatively, polymers including leaving groups react with protected amine salts such as potassium salt of methyl-tert-butyl imidodicarbonate (KNMeBoc) or the potassium salt of di-tert-butyl imidodicarbonate (KNBoc₂) followed by deprotecting the protected amine group. The purity of the polymers containing the terminal amines formed by these processes is greater than about 95% and preferably greater than 99%.

In alternative aspects, polymers having terminal carboxylic acid groups can be employed in the polymeric delivery systems described herein. Methods of preparing polymers having terminal carboxylic acids in high purity are described in U.S. patent application Ser. No. 11/328,662, the contents of which are incorporated herein by reference. The methods include first preparing a tertiary alkyl ester of a polyalkylene oxide followed by conversion to the carboxylic acid derivative thereof. The first step of the preparation of the PAO carboxylic acids of the process includes forming an intermediate such as t-butyl ester of polyalkylene oxide carboxylic acid. This intermediate is formed by reacting a PAO with a t-butyl haloacetate in the presence of a base such as potassium t-butoxide. Once the t-butyl ester intermediate has been formed, the carboxylic acid derivative of the polyalkylene oxide can be readily provided in purities exceeding 92%, preferably exceeding 97%, more preferably exceeding 99% and most preferably exceeding 99.5% purity.

### C. Bifunctional Linkers

Bifunctional linkers include amino acids or amino acid derivatives. The amino acids can be among naturally occurring and non-naturally occurring amino acids. Derivatives and analogs of the naturally occurring amino acids, as well as various art-known non-naturally occurring amino acids (D or L), hydrophobic or non-hydrophobic, are also contemplated to be within the scope of the invention. A suitable non-limiting list of the non-naturally occurring amino acids includes 2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4-aminobutyric acid, desmosine, 2,2-diaminopimelic acid, 2,3-diaminopropionic acid, N-ethylglycine, N-ethylasparagine, 3-hydroxyproline, 4-hydroxyproline, isodesmosine, allo-isoleucine, N-methylglycine, sarcosine, N-methyl-isoleucine, 6-N-methyl-lysine, N-methylvaline, norvaline, norleucine, and ornithine. Some preferred amino acid residues are selected from glycine, alanine, methionine or sarcosine, and more preferably, glycine.

Alternatively, L₁₋₃ and L′₁₋₃ are independently selected from among:

—[C(═O)]ᵥ(CR₂₂R₂₃)ₜ[C(═O)]_{v′}—,

—[C(═O)]ᵥ(CR₂₂R₂₃)ₜ—O[C(═O)]_{v′}—,

—[C(═O)]ᵥ(CR₂₂R₂₃)ₜ—NR₂₆[C(═O)]_{v′}—,

—[C(═O)]ᵥO(CR₂₂R₂₃)ₜ[C(═O)]_{v′}—,

—[C(═O)]ᵥO(CR₂₂R₂₃)ₜO[C(═O)]_{v′}—,

—[C(═O)]ᵥO(CR₂₂R₂₃)ₜNR₂₆[C(═O)]_{v′}—,

—[C(═O)]ᵥNR₂₁(CR₂₂R₂₃)ₜ[C(═O)]_{v′}—,

—[C(═O)]ᵥNR₂₁(CR₂₂R₂₃)ₜO[C(═O)]_{v′}—,

—[C(═O)]ᵥNR₂₁(CR₂₂R₂₃)ₜNR₂₆[C(═O)]_{v′}—,

—[C(═O)]ᵥ(CR₂₂R₂₃)ₜO—(CR₂₈R₂₉)_{t′}[C(═O)]_{v′}—,

—[C(═O)]ᵥ(CR₂₂R₂₃)ₜNR₂₆—(CR₂₈R₂₉)_{t′}[C(═O)]_{v′}—,

—[C(═O)]ᵥ(CR₂₂R₂₃)ₜS—(CR₂₈R₂₉)_{t′}[C(═O)]_{v′}—,

—[C(═O)]ᵥO(CR₂₂R₂₃)ₜO—(CR₂₈R₂₉)_{t′}[C(═O)]_{v′}—,

—[C(═O)]ᵥO(CR₂₂R₂₃)ₜNR₂₆—(CR₂₈R₂₉)_{t′}[C(═O)]_{v′}—,

—[C(═O)]ᵥO(CR₂₂R₂₃)ₜS—(CR₂₈R₂₉)_{t′}[C(═O)]_{v′}—,

—[C(═O)]ᵥNR₂₁(CR₂₂R₂₃)ₜO—(CR₂₈R₂₉)_{t′}[C(═O)]_{v′}—,

—[C(═O)]ᵥNR₂₁(CR₂₂R₂₃)ₜNR₂₆—(CR₂₈R₂₉)_{t′}[C(═O)]_{v′}—,

—[C(═O)]ᵥNR₂₁(CR₂₂R₂₃)ₜS—(CR₂₈R₂₉)_{t′}[C(═O)]_{v′}—,

—[C(═O)]ᵥ(CR₂₂R₂₃CR₂₈R₂₉O)ₜNR₂₆[C(═O)]_{v′}—,

—[C(═O)]ᵥ(CR₂₂R₂₃CR₂₈R₂₉O)ₜ[C(═O)]_{v′}—,

—[C(═O)]ᵥO(CR₂₂R₂₃CR₂₈R₂₉O)ₜNR₂₆[C(═O)]_{v′}—,

—[C(═O)]ᵥO(CR₂₂R₂₃CR₂₈R₂₉O)ₜ[C(═O)]_{v′}—,

—[C(═O)]ᵥNR₂₁(CR₂₂R₂₃CR₂₈R₂₉O)ₜNR₂₆[C(═O)]_{v′}—,

—[C(═O)]ᵥNR₂₁(CR₂₂R₂₃CR₂₈R₂₉O)ₜ[C(═O)]_{v′}—,

—[C(═O)]ᵥ(CR₂₂R₂₃CR₂₈R₂₉O)ₜ(CR₂₄R₂₅)_{t′}[C(═O)]_{v′}—,

—[C(═O)]ᵥO(CR₂₂R₂₃CR₂₈R₂₉O)ₜ(CR₂₄R₂₅)_{t′}[C(═O)]_{v′}—,

—[C(═O)]ᵥNR₂₁(CR₂₂R₂₃CR₂₈R₂₉O)ₜ(CR₂₄R₂₅)_{t′}[C(═O)]_{v′}—,

—[C(═O)]ᵥ(CR₂₂R₂₃CR₂₈R₂₉O)ₜ(CR₂₄R₂₅)_{t′}O[C(═O)]_{v′}—,

—[C(═O)]ᵥ(CR₂₂R₂₃)ₜ(CR₂₄R₂₅CR₂₈R₂₉O)_{t′}[C(═O)]_{v′}—,

—[C(═O)]ᵥ(CR₂₂R₂₃)ₜ(CR₂₄R₂₅CR₂₈R₂₉O)_{t′}NR₂₆[C(═O)]_{v′}—,

—[C(═O)]ᵥO(CR₂₂R₂₃CR₂₈R₂₉O)ₜ(CR₂₄R₂₅)_{t′}O[C(═O)]_{v′}—,

—[C(═O)]ᵥO(CR₂₂R₂₃)ₜ(CR₂₄R₂₅CR₂₈R₂₉O)_{t′}[C(═O)]_{v′}—,

—[C(═O)]ᵥO(CR₂₂R₂₃)ₜ(CR₂₄CR₂₅CR₂₈R₂₉O)_{t′}NR₂₆[C(═O)]_{v′}—,

—[C(═O)]ᵥNR₂₁(CR₂₂R₂₃CR₂₈R₂₉O)ₜ(CR₂₄R₂₅)_{t′}O[C(═O)]_{v′}—,

—[C(═O)]ᵥNR₂₁(CR₂₂R₂₃)ₜ(CR₂₄R₂₅CR₂₈R₂₉O)_{t′}[C(═O)]_{v′}—,

—[C(═O)]ᵥNR₂₁(CR₂₂R₂₃)ₜ(CR₂₄R₂₅CR₂₈R₂₉O)_{t′}NR₂₆[C(═O)]_{v′}—,

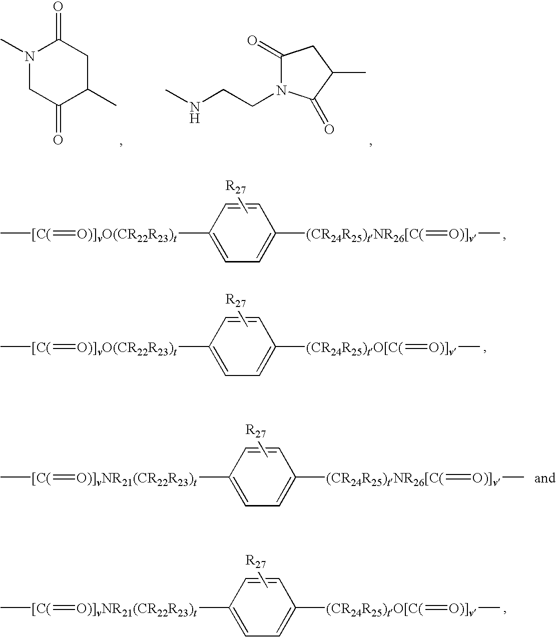

wherein:

R₂₁₋₂₉ are independently selected from among hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;

(t) and (t′) are independently zero or a positive integer, preferably zero or an integer from about 1 to about 12, more preferably an integer from about 1 to about 8, and most preferably 1 or 2; and

(v) and (v′) are independently zero or 1.

In some preferred embodiments, L₁₋₃ and L′₁₋₃ are independently selected from among:

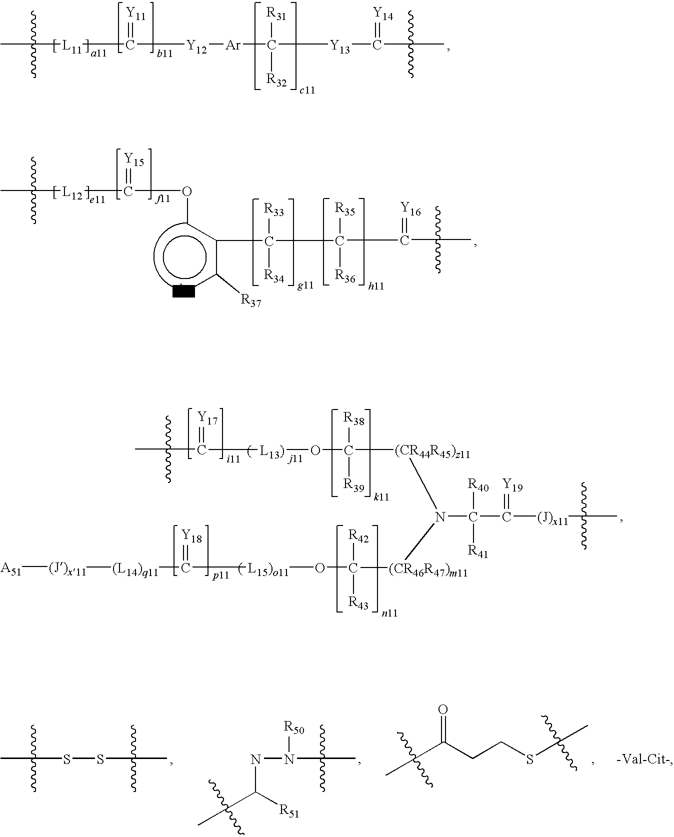

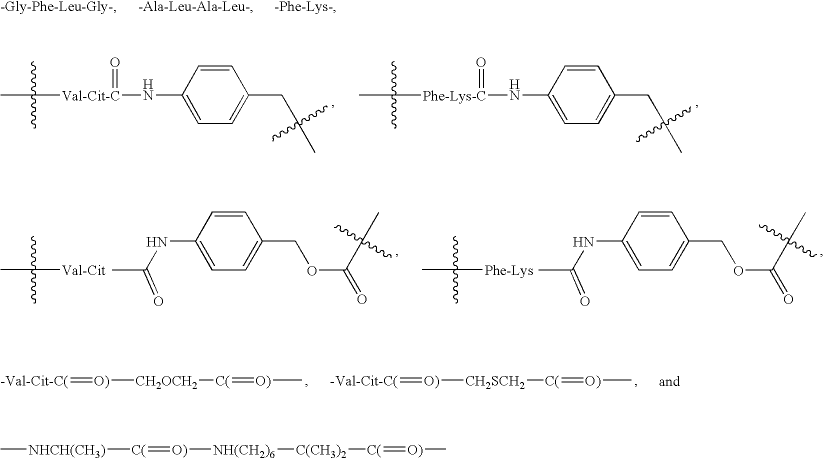

wherein,

Y₁₁₋₁₉ are independently O, S or NR₄₈;

R₃₁₋₄₈, R₅₀₋₅₁ and A₅₁ are independently selected from among hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;

Ar is an aryl or heteroaryl moiety;

L₁₁₋₁₅ are independently selected bifunctional spacers;

J and J′ are independently selected from selected from among moieties actively transported into a target cell, hydrophobic moieties, bifunctional linking moieties and combinations thereof;

(c11), (h11), (k11), (z11), (m11) and (n11) are independently selected positive integers, preferably 1;

(a11), (e11), (g11), (j11), (o11) and (q11) are independently either zero or a positive integer, preferably 1; and

(b11), (x11), (x′11), (f11), (i11) and (p11) are independently zero or one.

In more preferred embodiments, L₁₋₃ and L′₁₋₃ are independently selected from among:

—[C(═O)]ᵣNH(CH₂)₂CH═N—NHC(═O)—(CH₂)₂—,

—[C(═O)]ᵣNH(CH₂)₂(CH₂CH₂O)₂(CH₂)₂NH[C(═O)]_{r′}—,

—[C(═O)]ᵣNH(CH₂CH₂)(CH₂CH₂O)₂NH[C(═O)]_{r′}—,

—[C(═O)]ᵣNH(CH₂CH₂)ₛNH(CH₂CH₂)_{s′}[C(═O)]_{r′}—,

—[C(═O)]ᵣNH(CH₂CH₂)ₛS(CH₂CH₂)_{s′}[C(═O)]_{r′}—,

—[C(═O)]ᵣNH(CH₂CH₂)(CH₂CH₂O)[C(═O)]_{r′}—,

—[C(═O)]ᵣNH(CH₂CH₂)ₛO(CH₂CH₂)_{s′}[C(═O)]_{r′}—,

—[C(═O)]ᵣNH(CH₂CH₂O)(CH₂)NH[C(═O)]_{r′}—,

—[C(═O)]ᵣNH(CH₂CH₂O)₂(CH₂)[C(═O)]_{r′}—,

—[C(═O)]ᵣNH(CH₂CH₂O)ₛ(CH₂)_{s′}[C(═O)]_{r′}—,

—[C(═O)]ᵣNHCH₂CH₂NH[C(═O)]_{r′}—,

—[C(═O)]ᵣNH(CH₂CH₂)₂O[C(═O)]_{r′}—,

—[C(═O)]ᵣNH(CH₂CH₂O)[C(═O)]_{r′}—,

—[C(═O)]ᵣNH(CH₂CH₂O)₂[C(═O)]_{r′}—,

—[C(═O)]ᵣNH(CH₂)₃[C(═O)]_{r′}—,

—[C(═O)]ᵣO(CH₂CH₂O)₂(CH₂)[C(═O)]_{r′}—,

—[C(═O)]ᵣO(CH₂)₂NH(CH₂)₂[C(═O)]_{r′}—,

—[C(═O)]ᵣO(CH₂CH₂O)₂NH[C(═O)]_{r′}—,

—[C(═O)]ᵣO(CH₂)₂O(CH₂)₂[C(═O)]_{r′}—,

—[C(═O)]ᵣO(CH₂)₂S(CH₂)₂[C(═O)]_{r′}—,

—[C(═O)]ᵣO(CH₂CH₂)NH[C(═O)]_{r′}—,

—[C(═O)]ᵣO(CH₂CH₂)O[C(═O)]_{r′}—,

—[C(═O)]ᵣO(CH₂)₃NH[C(═O)]_{r′}—,

—[C(═O)]ᵣO(CH₂)₃O[C(═O)]_{r′}—,

—[C(═O)]ᵣO(CH₂)₃[C(═O)]_{r′}—,

—[C(═O)]ᵣCH₂NHCH₂[C(═O)]_{r′}—,

—[C(═O)]ᵣCH₂OCH₂[C(═O)]_{r′}—,

—[C(═O)]ᵣCH₂SCH₂[C(═O)]_{r′}—,

—[C(═O)]ᵣS(CH₂)₃[C(═O)]_{r′}—,

—[C(═O)]ᵣ(CH₂)₃[C(═O)]_{r′}—,

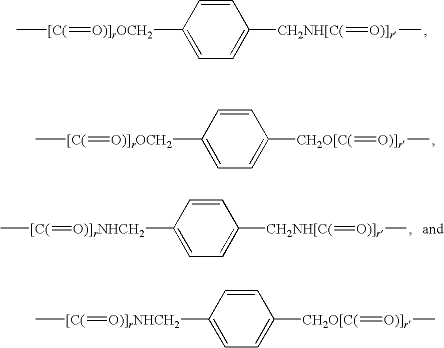

wherein, (r) and (r′) are independently zero or 1.

In a further embodiment and as an alternative, L₁₋₃ and L′₁₋₃ include structures corresponding to those shown above but having vinyl, residues of sulfone, amino, carboxy, mercapto, hydrazide, carbazate and the like instead of maleimidyl.

### D. R₉₋₁₀ and R′₉₋₁₀ Groups

### 1. Leaving Groups and Functional Groups

In some aspects, suitable leaving groups include, without limitations halogen (Br, Cl), activated carbonate, carbonyl imidazole, cyclic imide thion, isocyanate, N-hydroxysuccinimidyl, para-nitrophenoxy, N-hydroxyphtalimide, N-hydroxybenzotriazolyl, imidazole, tosylate, mesylate, tresylate, nosylate, C₁-C₆ alkyloxy, C₁-C₆ alkanoyloxy, arylcarbonyloxy, ortho-nitrophenoxy, N-hydroxybenzotriazolyl, imidazole, pentafluorophenoxy, 1,3,5-trichlorophenoxy, and 1,3,5-trifluorophenoxy or other suitable leaving groups as will be apparent to those of ordinary skill.

For purposes of the present invention, leaving groups are to be understood as those groups which are capable of reacting with a nucleophile found on the desired target, i.e. a biologically active moiety, a diagnostic agent, a targeting moiety, a bifunctional spacer, intermediate, etc. The targets thus contain a group for displacement, such as OH, NH₂ or SH groups found on proteins, peptides, enzymes, naturally or chemically synthesized therapeutic molecules such as doxorubicin, and spacers such as mono-protected diamines.

In some preferred embodiments, functional groups to link the polymeric transport systems to biologically active moieties include maleimidyl, vinyl, residues of sulfone, amino, carboxy, mercapto, hydrazide, carbazate and the like which can be further conjugated to a biologically active group.

In yet some preferred embodiments of the invention, R₉₋₁₀ and R′₉₋₁₀ can be selected from among H, OH, methoxy, tert-butoxy, N-hydroxysuccinimidyl and maleimidyl.

### 2. Biologically Active Moieties

In some aspects of the invention, biologically active moieties include amine-, hydroxyl-, or thiol-containing compounds. A non-limiting list of such suitable compounds includes organic compounds, enzymes, proteins, polypeptides, antibodies, monoclonal antibodies, single chain antibodies or oligonucleotides, etc. Organic compounds include, without limitation, moieties such as camptothecin and analogs such as SN38 and irinotecan, and related topoisomerase I inhibitors, taxanes and paclitaxel derivatives, nucleosides including AZT, anthracycline compounds including daunorubicin, doxorubicin; p-aminoaniline mustard, melphalan, Ara-C (cytosine arabinoside) and related anti-metabolite compounds, e.g., gemcitabine, etc. Alternatively, biologically active moieties can include cardiovascular agents, anti-neoplastic, anti-infective, anti-fungal such as nystatin and amphotericin B, anti-anxiety agents, gastrointestinal agents, central nervous system-activating agents, analgesic, fertility agents, contraceptive agents, anti-inflammatory agents, steroidal agents, anti-urecemic agents, vasodilating agents, and vasoconstricting agents, etc. It is to be understood that other biologically active materials not specifically mentioned but having suitable amine-, hydroxyl- or thiol-containing groups are also intended and are within the scope of the present invention.

In another aspect of the invention, the biologically active compounds are suitable for medicinal or diagnostic use in the treatment of animals, e.g., mammals, including humans, for conditions for which such treatment is desired.

The only limitations on the types of the biologically active moieties suitable for inclusion herein is that there is available at least one amine-, hydroxyl-, or thiol-containing position which can react and link with a carrier portion and that there is not substantial loss of bioactivity in the form of conjugated to the polymeric delivery systems described herein. Alternatively, parent compounds suitable for incorporation into the polymeric transport conjugate compounds of the invention, may be active after hydrolytic release from the linked compound, or not active after hydrolytic release but which will become active after undergoing a further chemical process/reaction. For example, an anticancer drug that is delivered to the bloodstream by the polymeric transport system, may remain inactive until entering a cancer or tumor cell, whereupon it is activated by the cancer or tumor cell chemistry, e.g., by an enzymatic reaction unique to that cell.

A further aspect of the invention provides the conjugate compounds optionally prepared with a diagnostic tag linked to the polymeric delivery system described herein, wherein the tag is selected for diagnostic or imaging purposes. Thus, a suitable tag is prepared by linking any suitable moiety, e.g., an amino acid residue, to any art-standard emitting isotope, radio-opaque label, magnetic resonance label, or other non-radioactive isotopic labels suitable for magnetic resonance imaging, fluorescence-type labels, labels exhibiting visible colors and/or capable of fluorescing under ultraviolet, infrared or electrochemical stimulation, to allow for imaging tumor tissue during surgical procedures, and so forth. Optionally, the diagnostic tag is incorporated into and/or linked to a conjugated therapeutic moiety, allowing for monitoring of the distribution of a therapeutic biologically active material within an animal or human patient.

In a still further aspect of the invention, the inventive tagged conjugates are readily prepared, by art-known methods, with any suitable label, including, e.g., radioisotope labels. Simply by way of example, these include ¹³¹Iodine, ¹²⁵Iodine, ^{99m}Technetium and/or ¹¹¹Indium to produce radioinuuno-scintigraphic agents for selective uptake into tumor cells, in vivo. For instance, there are a number of art-known methods of linking peptide to Tc-99m, including, simply by way of ex-ample, those shown by U.S. Pat. Nos. 5,328,679; 5,888,474; 5,997,844; and 5,997,845, incorporated by reference herein.

### 3. Targeting Groups

In some aspects, the compounds described herein include targeting groups. The targeting groups include receptor ligands, an antibodies or antibody fragments, single chain antibodies, targeting peptides, targeting carbohydrate molecules or lectins. Targeting groups enhance binding or uptake of the compounds described herein a target tissue and cell population. For example, a non-limiting list of targeting groups includes vascular endothelial cell growth factor, FGF2, somatostatin and somatostatin analogs, transferrin, melanotropin, ApoE and ApoE peptides, von Willebrand's Factor and von Willebrand's Factor peptides, adenoviral fiber protein and adenoviral fiber protein peptides, PD1 and PD1 peptides, EGF and EGF peptides, RGD peptides, folate, etc. In another aspect of the invention the targeting groups include monoclonal antibody, single chain antibody, biotin, cell adhesion peptides, cell penetrating peptides (CPPs), fluorescent compounds, radio-labeled compounds, and aptamers. In a still further aspect of the invention the targeting agent can include Selectin, TAT, Penetratin, PolyArg, and folic acid.

### E. Synthesis of the Polymeric Delivery Systems

Generally, the methods of preparing the compounds described herein include reacting the polymer with the branching moiety to form a polymer with a branching unit. In one aspect of the invention, methods of preparing compounds described herein include:

reacting a polymeric compound of Formula (III):

A₁-R₁-M₁ (III)

with a compound of Formula (IV) containing a branching moiety in a protected form:

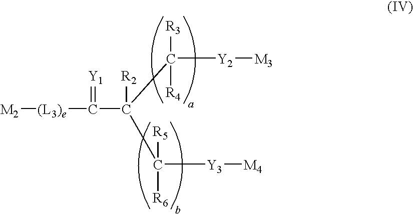

under conditions sufficient to form a compound of the formula (V):

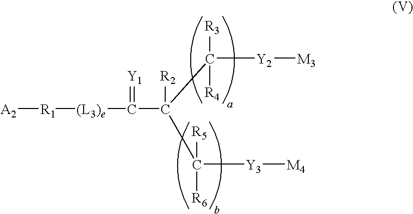

wherein, R₁ is a substantially non-antigenic water-soluble polymer;

A₁ is a capping group or M₁;

A₂ is a capping group or

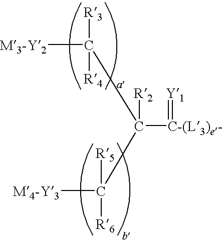

M₁ is —OH, SH, or —NHR₃₀;

M₂ is OH or a leaving group selected from among halogens, activated carbonates, activated ester, isocyanate, N-hydroxysuccinimidyl, tosylate, mesylate, tresylate, nosylate, ortho-niitrophenoxy and imidazole;

M₃₋₄ and M′₃₋₄ are independently selected protecting groups selected from among t-Boc (tert-butyloxycarbonyl), Cbz (carbobenzyloxy) and TROC (trichloroethoxycarbonyl);

L₃ and L′₃ are independently selected bifunctional linkers;

Y₁ and Y′₁, are independently O, S, or NR₂₀;

Y₂₋₃ and Y′₂₋₃ are independently O, S, SO, SO₂ or NR₇;

R₂₋₇, R′₂₋₆, R₂₀ and R₃₀ are independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy;

(a) and (a′) are independently zero or a positive integer, preferably zero or an integer from 1 to 3 and more preferably zero;

(b) and (b′) are independently a positive integer, preferably from 1 to 10, more preferably 2 to 6 and most preferably 4; and

(e) and (e′) are independently zero or 1.

The resulting compound of Formula (V) can be deprotected by treatment with a strong acid such as trifluoroacetic acid (TFA) or other haloacetic acid, HCl, sulfuric acid, etc. or by using catalytic hydrogenation to form a compound of Formula (V′):

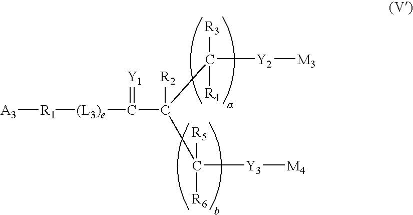

wherein:

A₃ is a capping group or

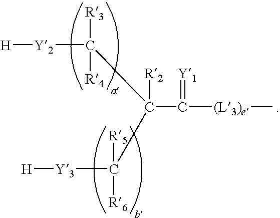

Alternatively, it is also contemplated that method can include reacting the resulting unprotected amino terminal group further with a compound of Formula (VI):

M₅-(L″₁)_{c}-R″₉ (VI)

under conditions sufficient to form a compound of Formula (VII)

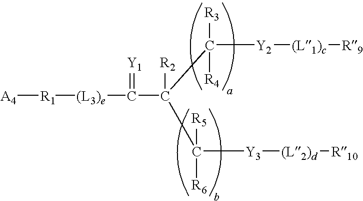

wherein

A₄ is a capping group or

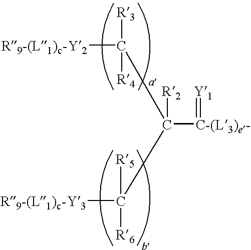

each R″₉ is independently a targeting group, a diagnostic agent or a biologically active moiety;

M₅ is —OH or a leaving group;

each L″₁ is independently a bifunctional linker; and

each (c) is independently zero or 1.

Attachment of the branching moiety to the polymer portion or conjugation of the polymeric system containing branching moiety with the compound of Formula (VI) is preferably carried out in the presence of a coupling agent. A non-limiting list of suitable coupling agents include 1,3-diisopropylcarbodiimide (DIPC), any suitable dialkyl carbodiimides, 2-halo-1-alkyl-pyridinium halides, (Mukaiyama reagents), 1-(3-dimethylaminopropyl)-3-ethyl carbodiimide (EDC), propane phosphonic acid cyclic anhydride (PPACA), and phenyl dichlorophosphates, etc. which are available, for example from commercial sources such as Sigma-Aldrich Co., or synthesized using known techniques.

Preferably, the reactions are carried out in an inert solvent such as methylene chloride, chloroform, DMF or mixtures thereof. The reactions can be preferably conducted in the presence of a base, such as dimethylaminopyridine (DMAP), diisopropylethylamine, pyridine, triethylamine, etc. to neutralize any acids generated. The reactions can be carried out at a temperature from about 0° C. up to about 22° C. (room temperature).

Some particular embodiments prepared by the methods described herein include:

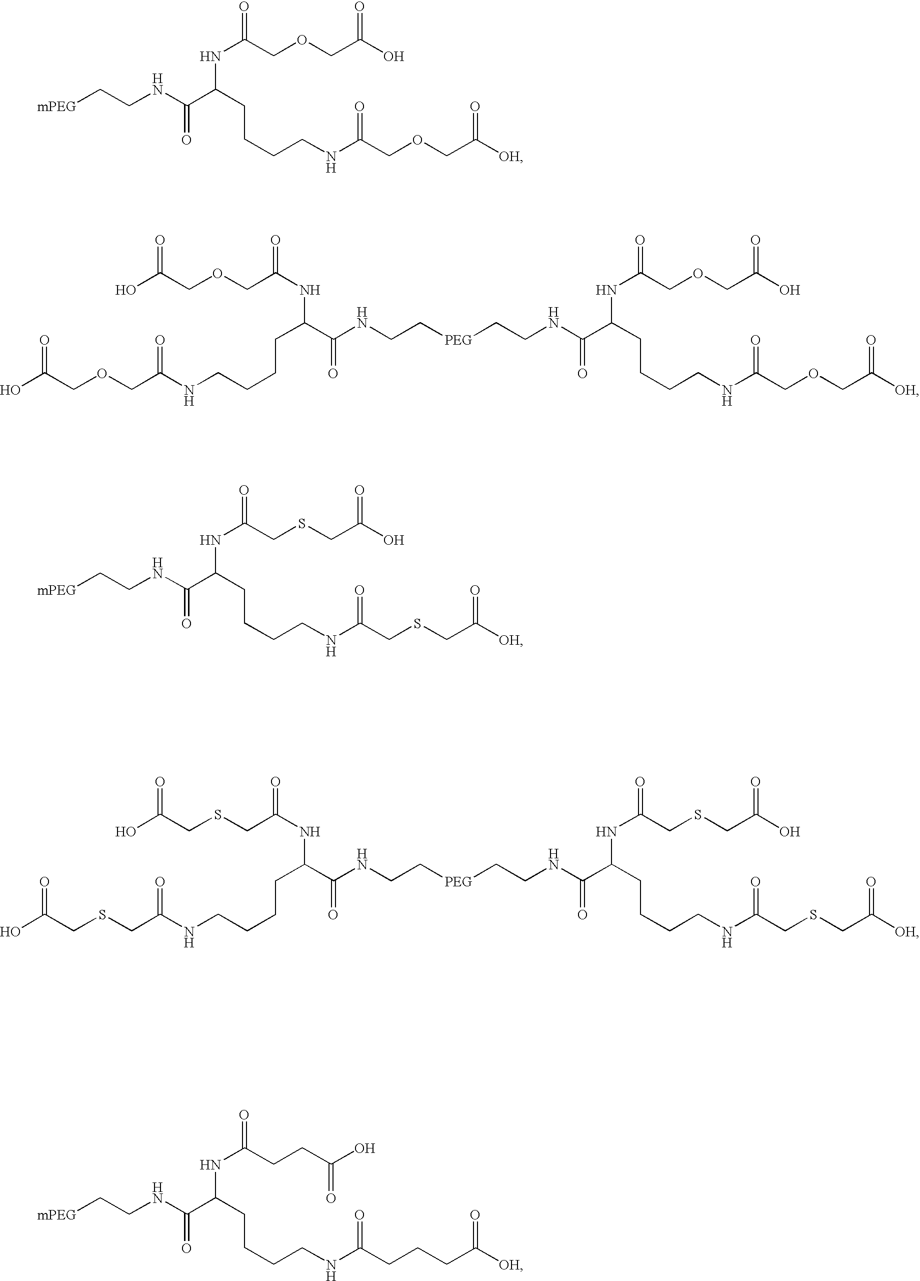

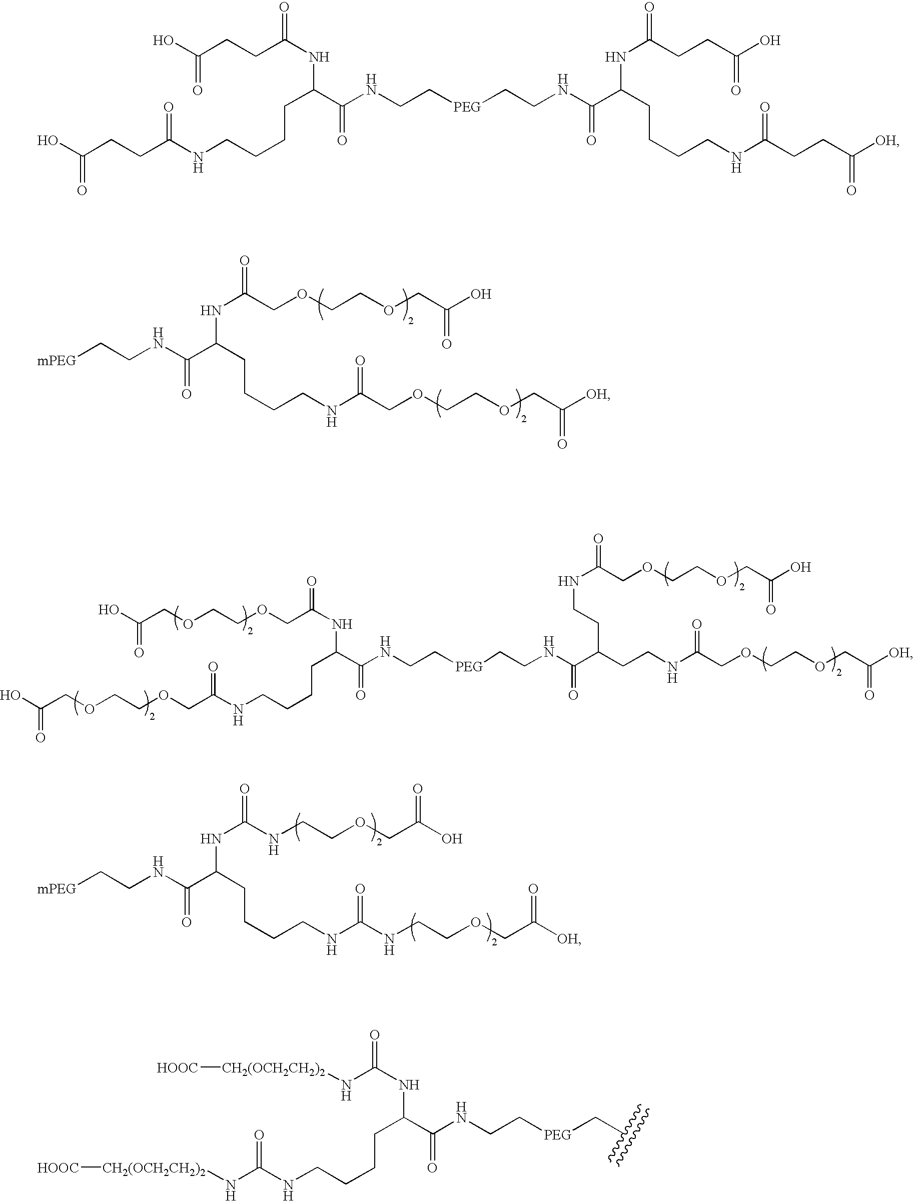

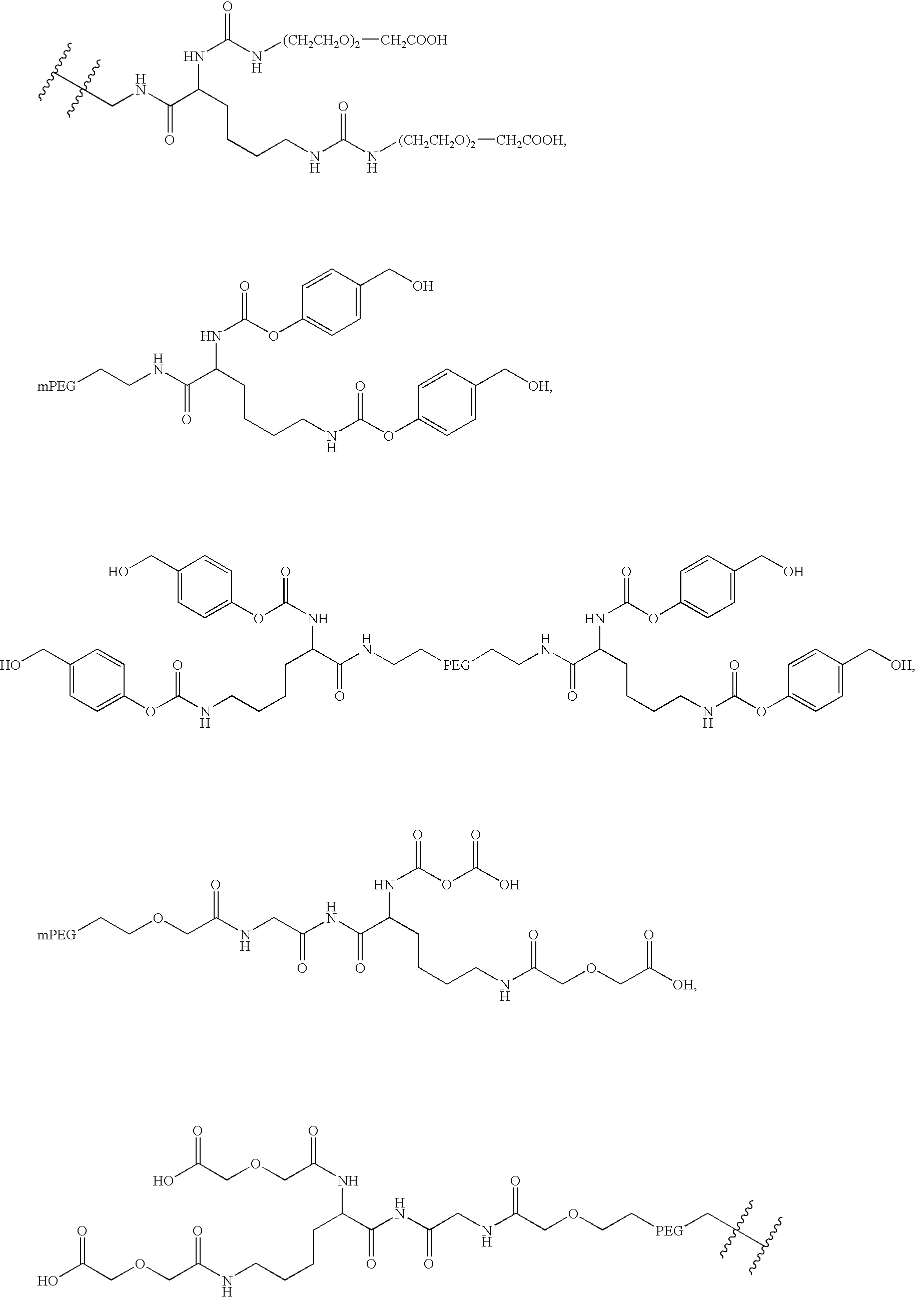

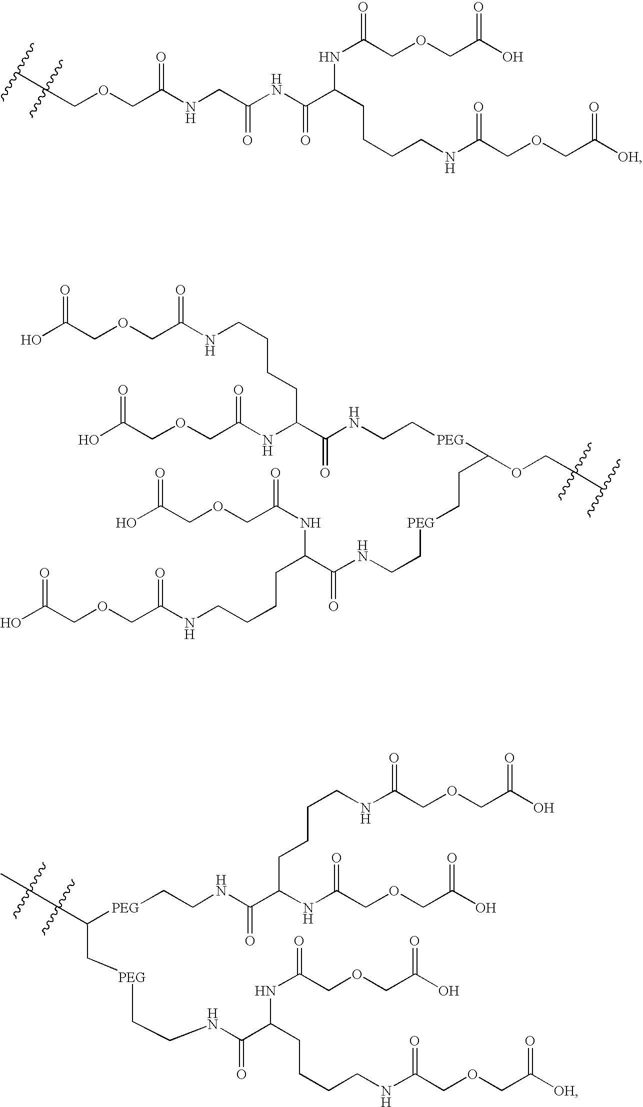

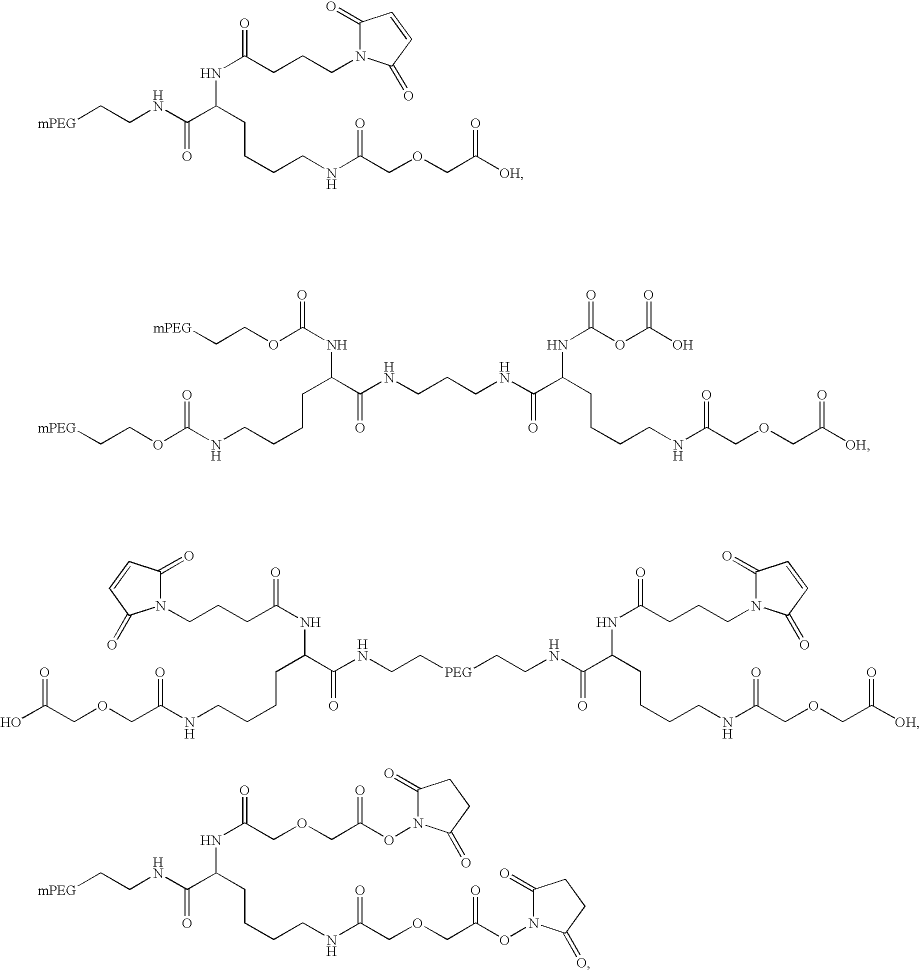

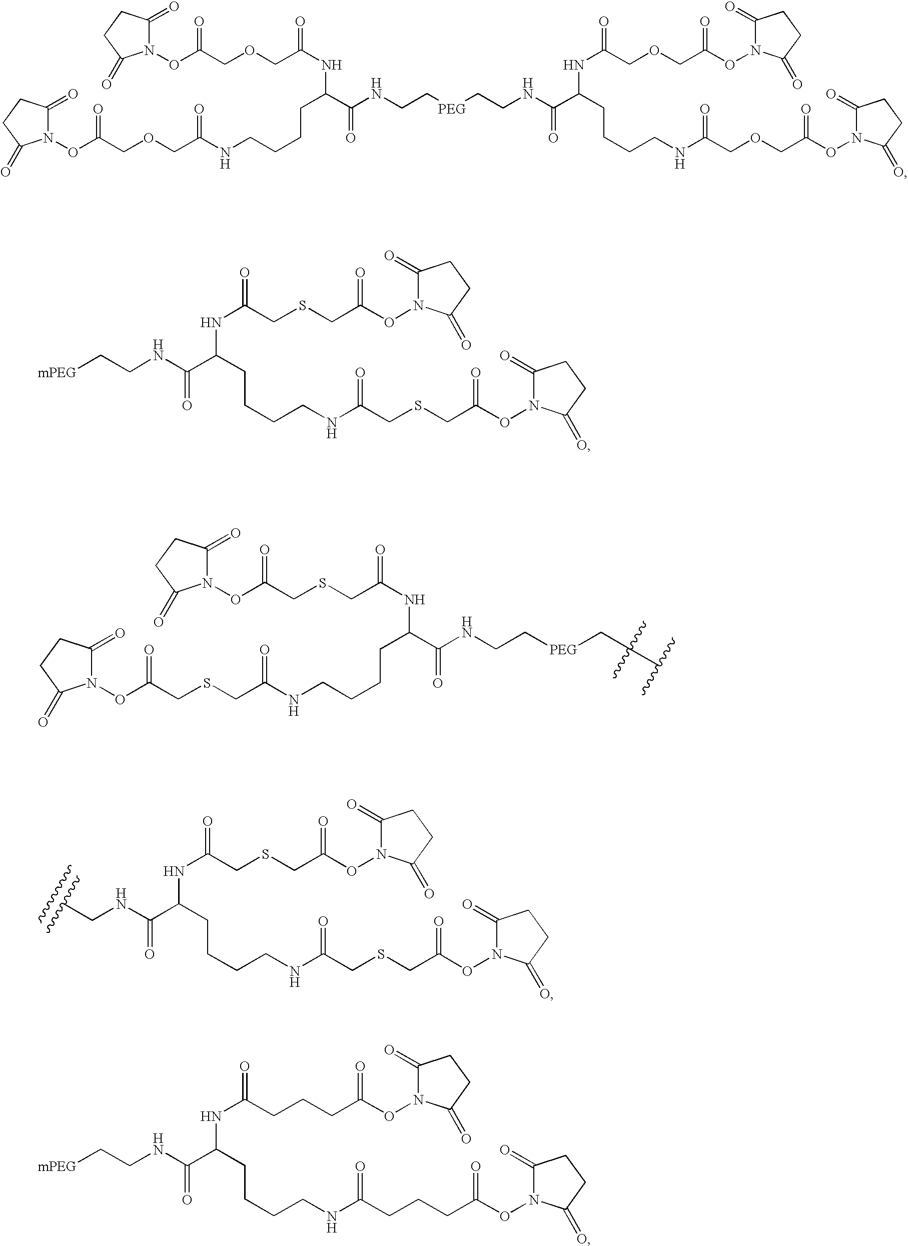

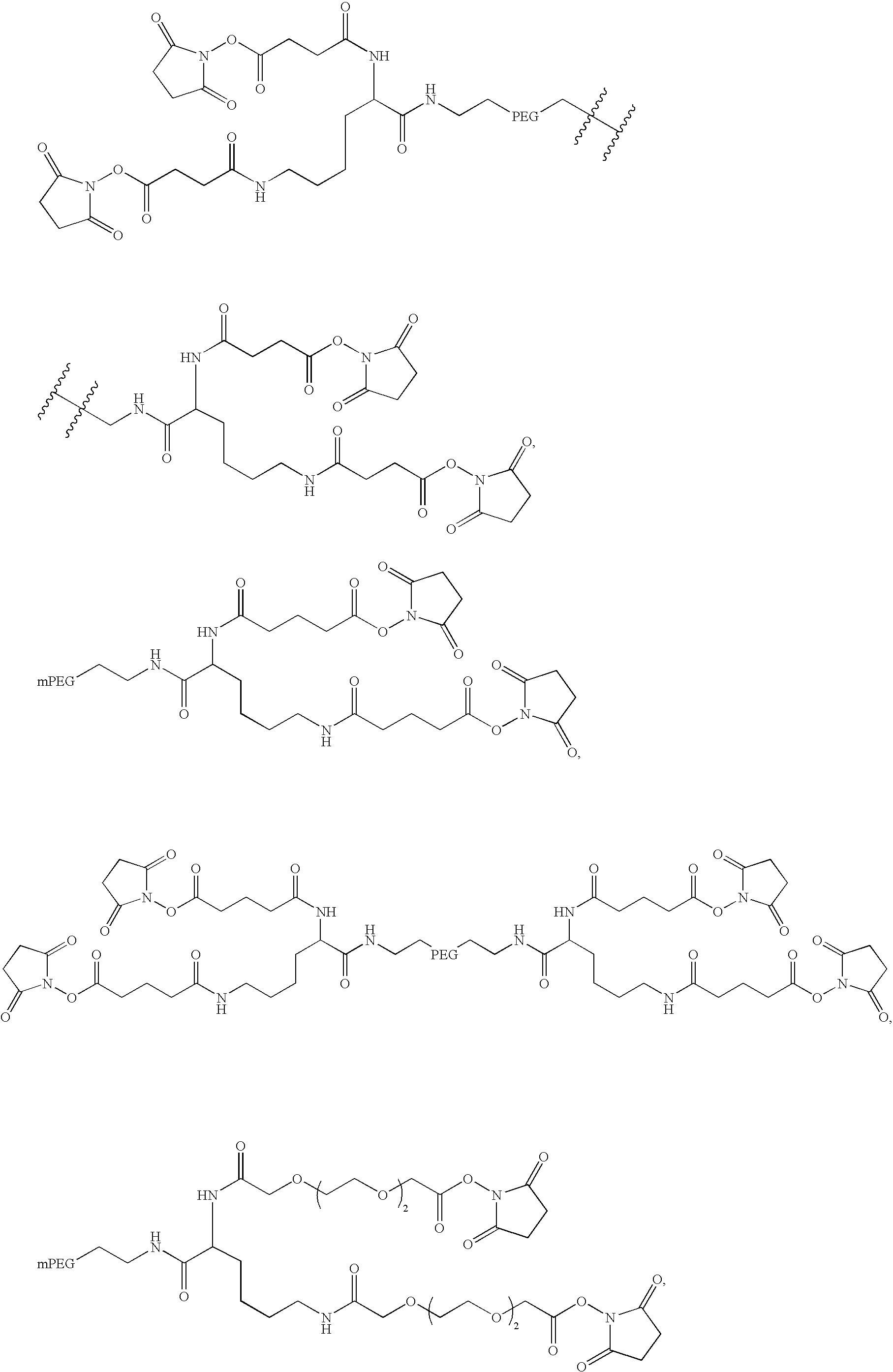

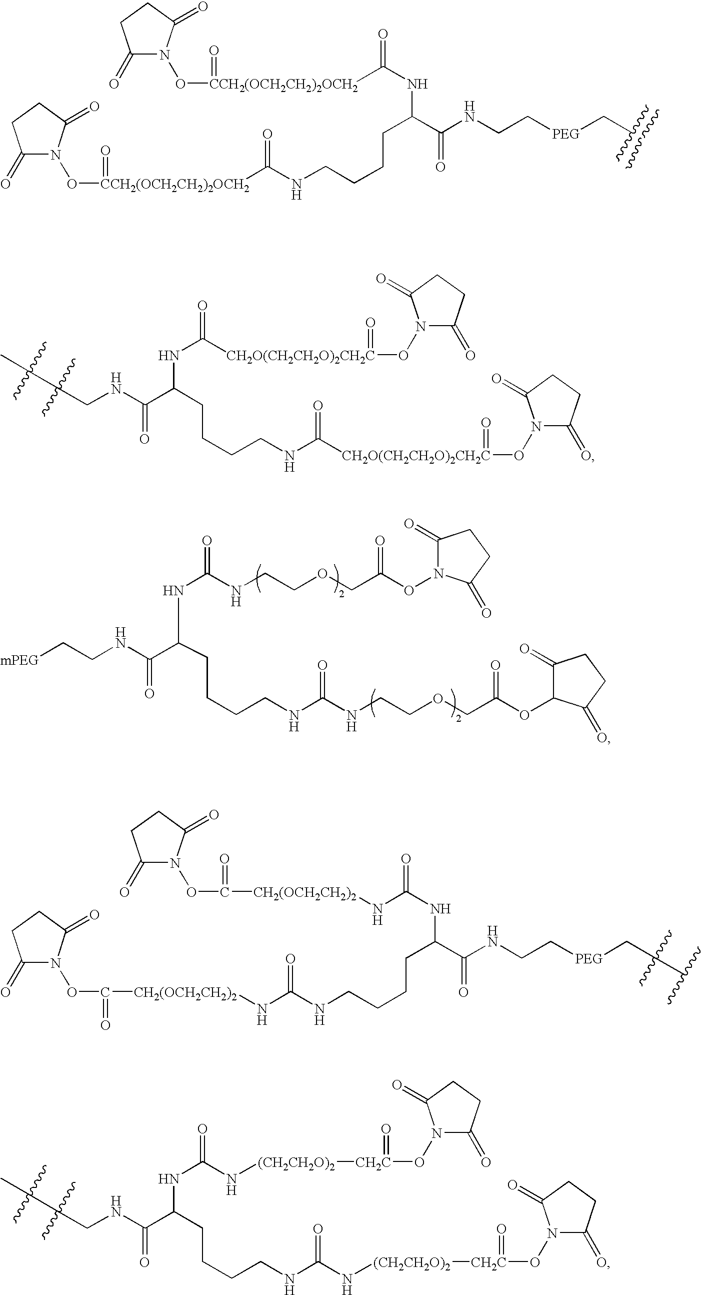

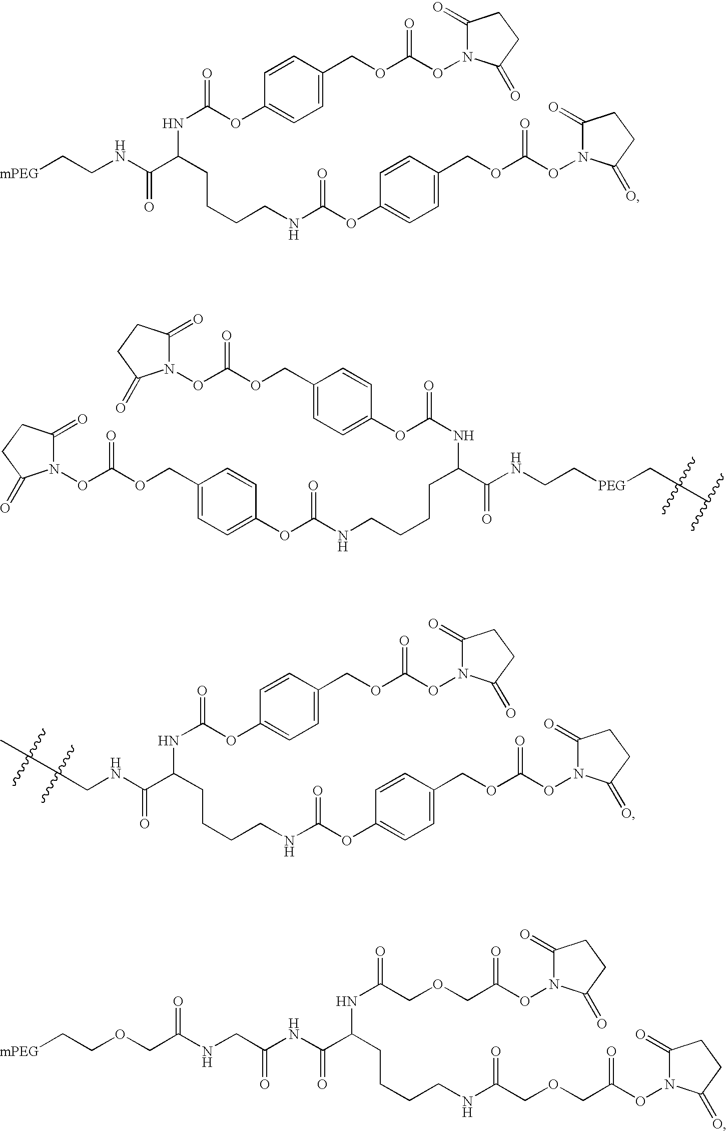

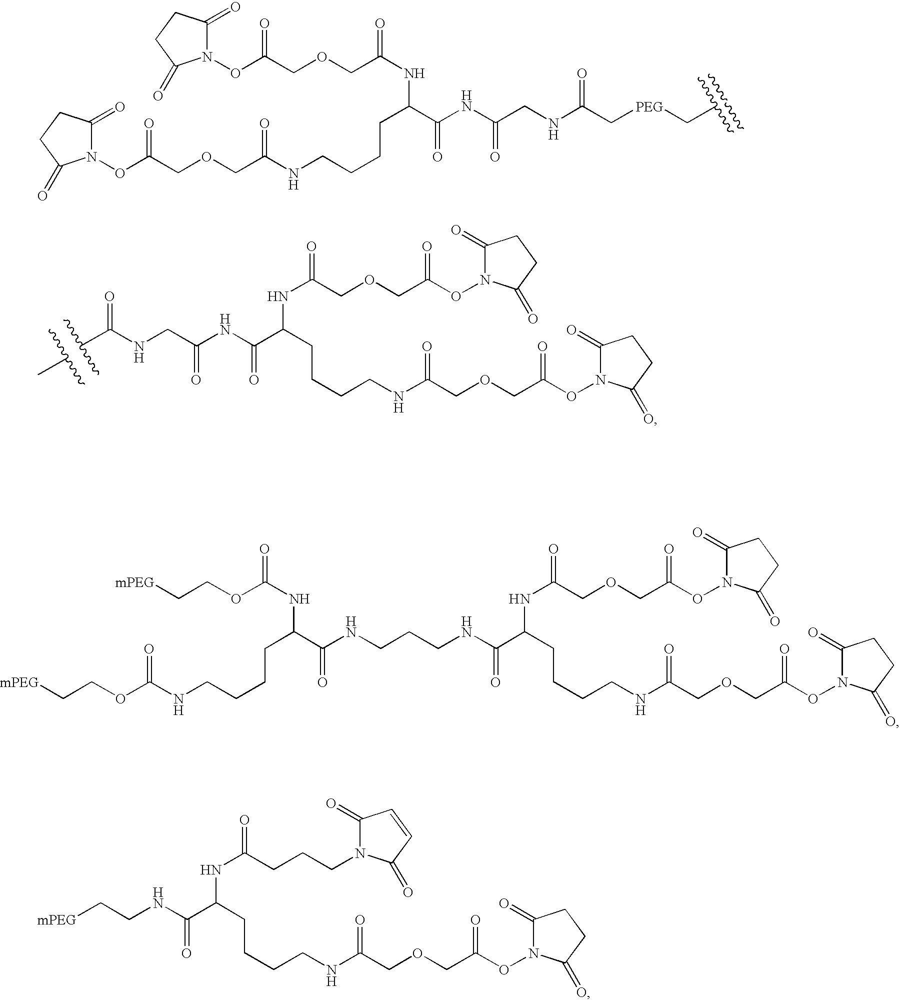

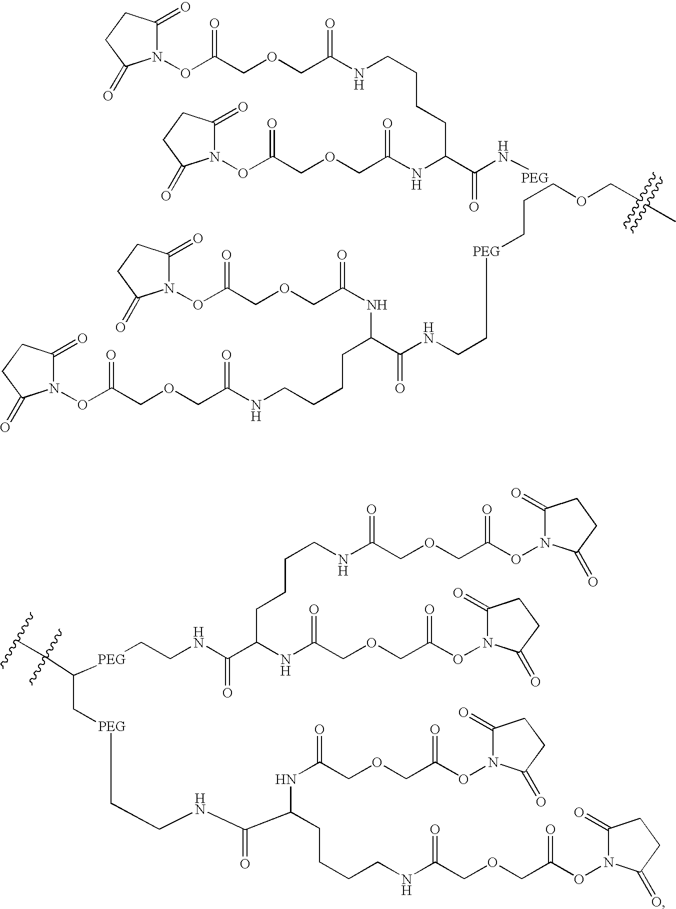

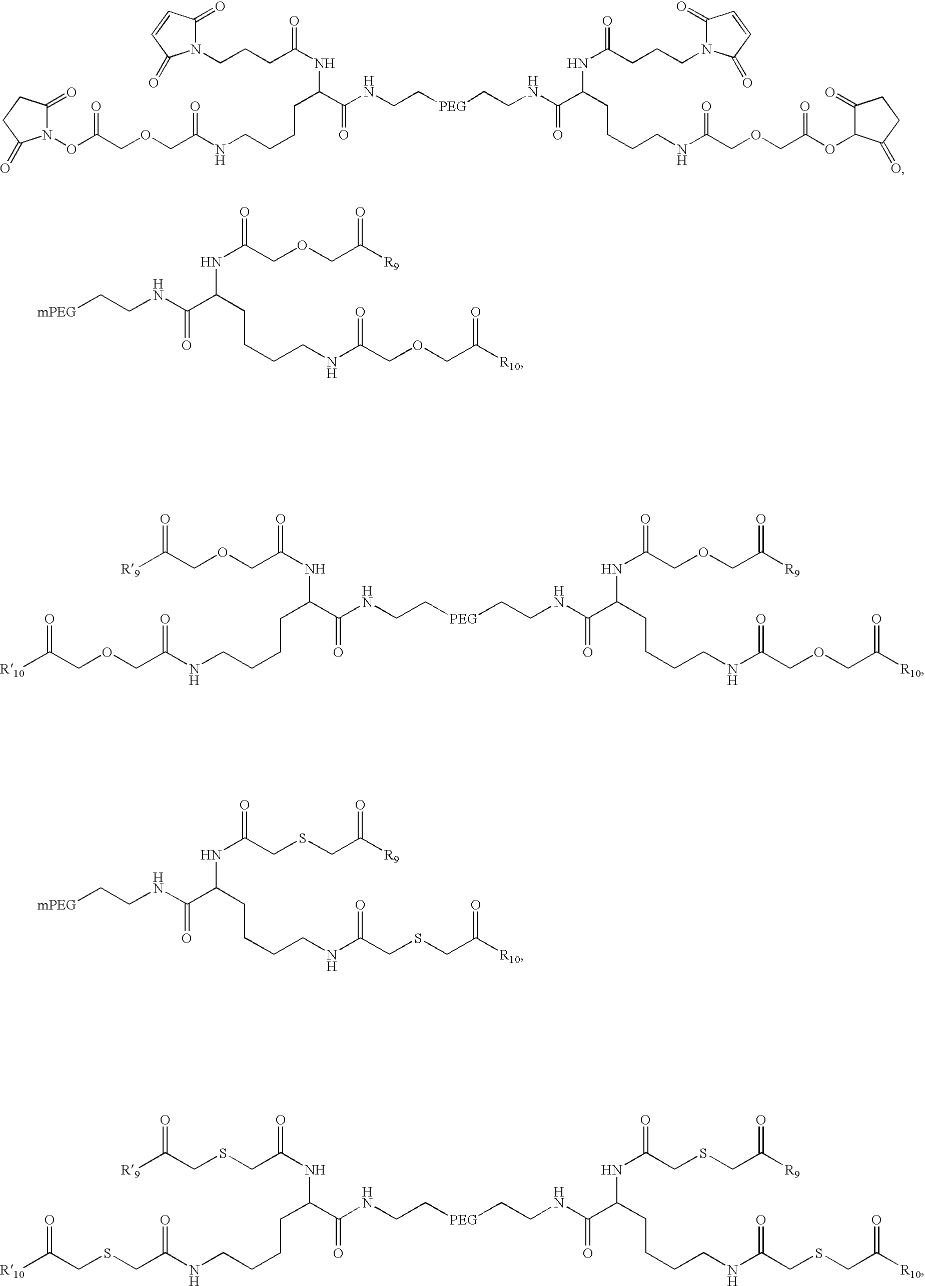

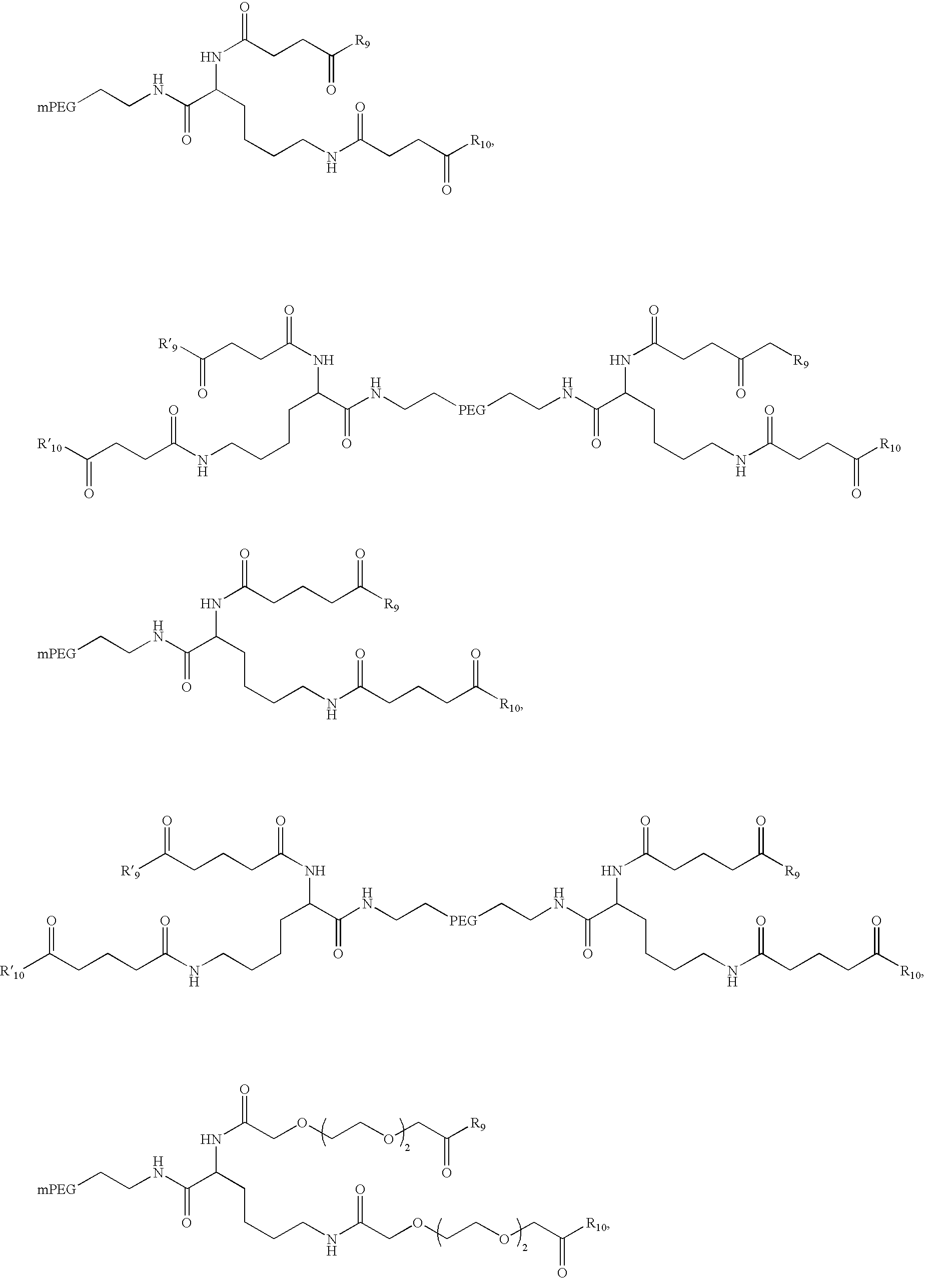

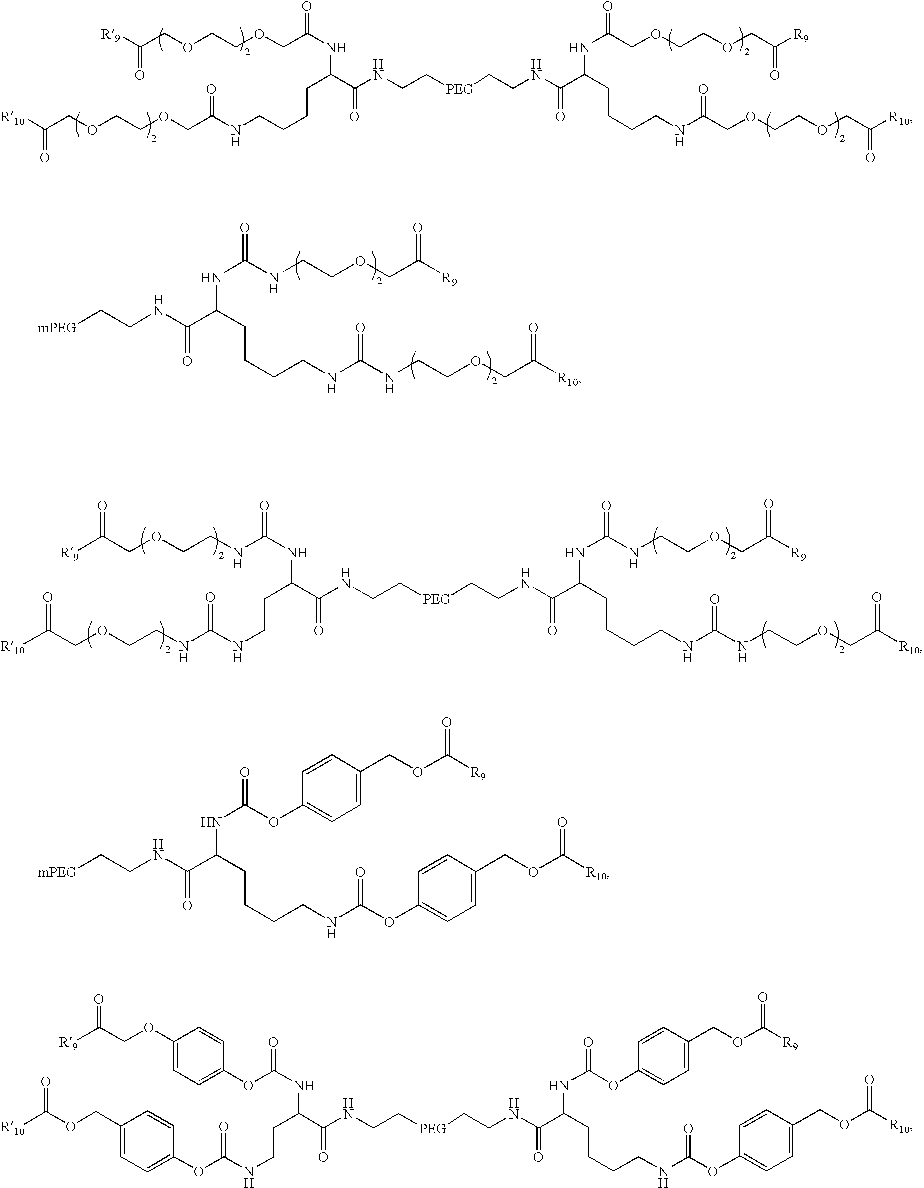

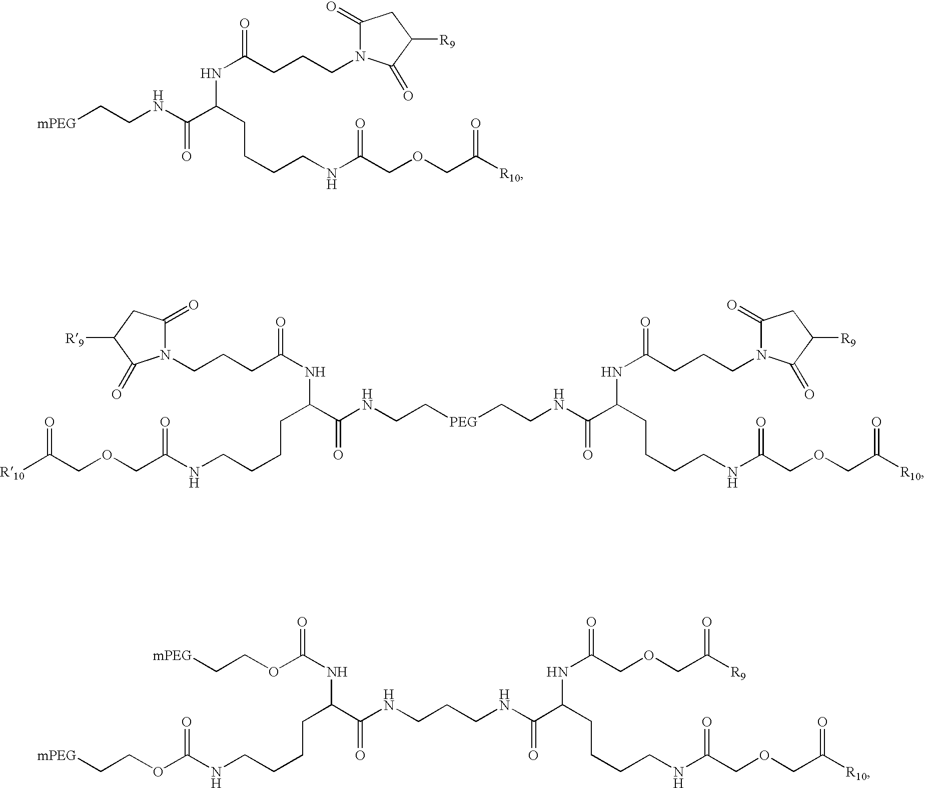

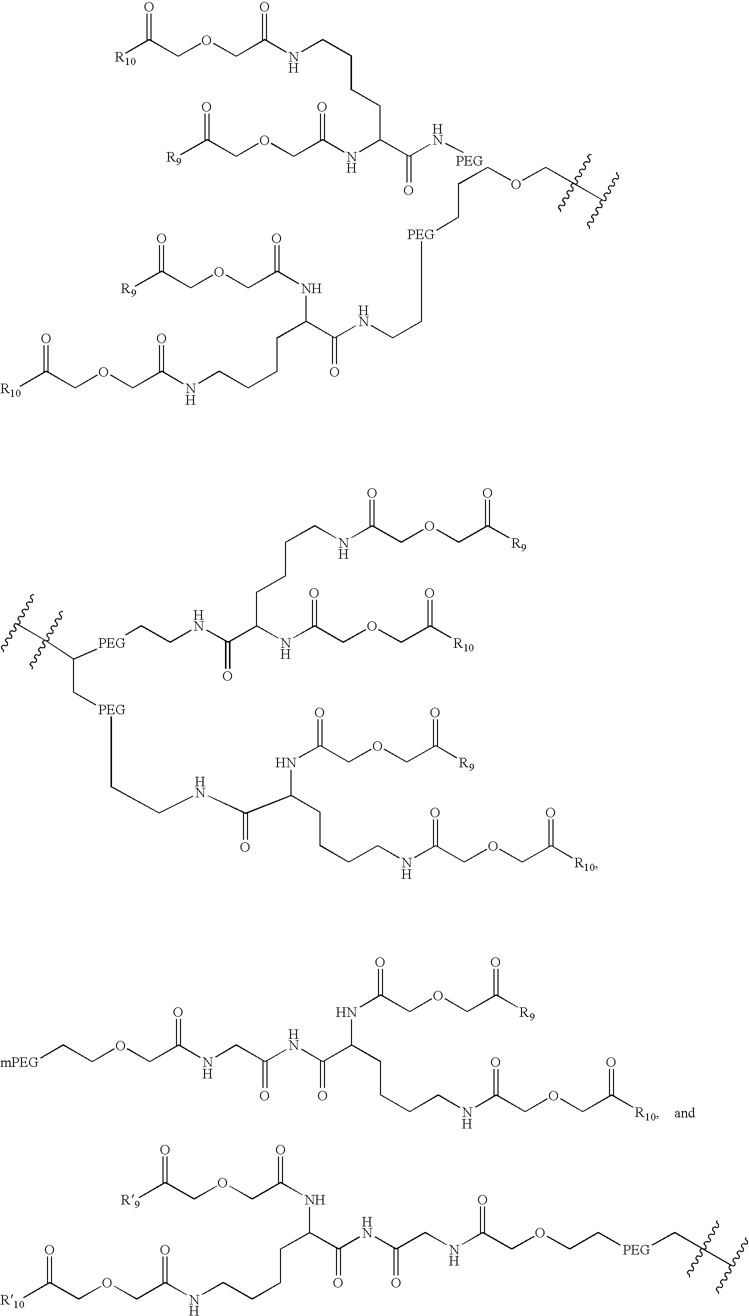

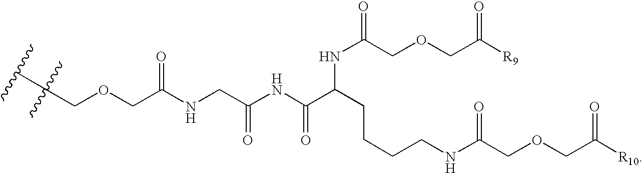

wherein:

mPEG has the formula CH₃O(CH₂CH₂O)ₙ—;

PEG has the formula —O(CH₂CH₂O)ₙ—,

(n) is an integer from about 10 to about 2,300; and

R₉₋₁₀ and R′₉₋₁₀ are independently selected from among targeting groups, diagnostic agents and biologically active moieties

### F. Methods of Treatment

Another aspect of the present invention provides methods of treatment for various medical conditions in mammals. The methods include administering, to the mammal in need of such treatment, an effective amount of a compound described herein. The polymeric conjugate compounds are useful for, among other things, treating diseases which are similar to those which are treated with the parent compound, e.g. enzyme replacement therapy, neoplastic disease, reducing tumor burden, preventing metastasis of neoplasms and preventing recurrences of tumor/neoplastic growths in mammals.

The amount of the polymeric conjugate that is administered will depend upon the amount of the parent molecule included therein. Generally, the amount of polymeric conjugate used in the treatment methods is that amount which effectively achieves the desired therapeutic result in mammals. Naturally, the dosages of the various polymeric conjugate compounds will vary somewhat depending upon the parent compound, molecular weight of the polymer, rate of in viva hydrolysis, etc. Those skilled in the art will determine the optimal dosing of the polymeric transport conjugates selected based on clinical experience and the treatment indication. Actual dosages will be apparent to the artisan without undue experimentation.

The compounds of the present invention can be included in one or more suitable pharmaceutical compositions for administration to mammals. The pharmaceutical compositions may be in the form of a solution, suspension, tablet, capsule or the like, prepared according to methods well known in the art. It is also contemplated that administration of such compositions may be by the oral and/or parenteral routes depending upon the needs of the artisan. A solution and/or suspension of the composition may be utilized, for example, as a carrier vehicle for injection or infiltration of the composition by any art known methods, e.g., by intravenous, intramuscular, intraperitoneal, subcutaneous injection and the like. Such administration may also be by infusion into a body space or cavity, as well as by inhalation and/or intranasal routes. In preferred aspects of the invention, however, the polymeric conjugates are parenterally administered to mammals in need thereof.

### EXAMPLES

The following examples serve to provide further appreciation of the invention but are not meant in any way to restrict the scope of the invention. The bold-faced numbers recited in the Examples correspond to those shown in Figs. Abbreviations are used throughout the examples such as, DCM (dichloromethane), DIEA (diisopropylethylamine), DMAP (4-dimethylaminopyridine), DMF (N,N′-dimethylformamide), DSC (disuccinimidyl carbonate), EDC (1-(3-dimethylaminopropyl)-3-ethyl carbodiimide), IPA (isopropanol), NTS (N-hydroxysuccinimide), PEG (polyethylene glycol), SCA-SH (single-chain antibody), SN38 (7-ethyl-10-hydroxy-camptothecin), TBDPS (tert-butyl-dipropylsilyl), and TEA (triethylamine).

General Procedures. All reactions are run under an atmosphere of dry nitrogen or argon. Commercial reagents are used without further purification. All PEG compounds are dried under vacuum or by azeotropic distillation from toluene prior to use. ¹H NMR spectra were obtained at 300 MHz and ¹³C NMR spectra were obtained at 75.46 MHz using a Varian Mercury®300 NMR spectrometer and deuterated chloroform as the solvents unless otherwise specified. Chemical shifts (δ) are reported in parts per million (ppm) downfield from tetramethylsilane (TMS).
HPLC Method. The reaction mixtures and the purity of intermediates and final products are monitored by a Beckman Coulter System Gold® HPLC instrument. It employs a ZORBAX® 300SB C8 reversed phase column (150×4.6 mm) or a Phenomenex Jupiter® 300A C18 reversed phase column (150×4.6 mm) with a 168 Diode Array UV Detector, using a gradient of 10-90% of acetonitrile in 0.05% trifluoroacetic acid (TFA) at a flow rate of 1 mL/min.)

### Example 1

### PEG-[Lys (Boc)₂]₂, Compound (3)

PEG-diamine (compound 1, Mw. 20 kDa, 25 g, 1.25 mmol) was azeotroped and toluene was removed in vacuo to dryness. Dissolved in 200 mL of DCM and Boc-Lys-Boc (compound 2, 2.638 g, 5 mmol) and DMAP (610 mg, 5 mmol) were added and the reaction mixture was cooled to 0° C. for 15 minutes before the addition of EDC (958 mg, 5 mmol). The reaction mixture was allowed to warm to room temperature with stirring overnight. The solvent was removed in vacuo to dryness and the residue was recrystallized form 2-Propanol to give 14 g of the product: ¹³C NMR δ 171.30, 78.3, 53.44, 39.08, 38.27, 31.59, 28.72, 27.63, 27.52, 21.71.

### Example 2

### PEG-[Lys (NH₂)₂], Compound (4)

Compound 3 (14 g) was dissolved in 240 mL of TFA/DCM (1:1) mixture and stirred for 4 hours at room temperature. The reaction mixture was concentrated in vacuo and the residue was precipitated by adding ethyl ether and the solvent was decanted. The solid was dissolved 60 mL of 0.1 M NaHCO₃ and extracted with DCM until aqueous layer becomes clear. The organic layer was dried over anhydrous MgSO₄ and the solvent was removed in vacuo to give the crude product which was recrystallized from 2-propanol to give 13 g of the product: ¹³C NMR δ 174.4, 53.79, 39.13, 37.9, 33.55, 26.90, 21.71.

### Example 3

### SCH AF-DGA-OH, Compound (7a)

Compound SCH—OH (compound SCH AF, 5.0 g, 7.135 mmol), DMAP (3.49 g, 28.5 mmol), and diglycolic anhydride (compound 6, 1.66 g, 14.3 mmol) were dissolved in 200 mL anhydrous DCM and stirred for 2 hours. The solution was then washed by 100 mL of 0.1 N HCl four times and dried over anhydrous MgSO₄. The solution was filtered and the solvent was removed in vacuo. The residue was dried under vacuum overnight to give the product (5.61 g, 6.87 mmol, 96%): ¹³C NMR δ 10.23, 17.11, 22.07, 37.33, 38.65, 48.73, 50.69, 53.34, 55.88, 60.18, 68.03, 68.18, 68.75, 70.53, 71.96, 83.76 (J_{CF}=4 Hz), 104.46 (J_{CF}=261 Hz), 111.18 (J_{CF}=20 Hz), 115.03, 116.51, 118.66, 123.53, 125.11 (J_{CF}=12 Hz), 125.39, 128.44 (J_{CF}=7 Hz), 134.64, 144.32, 144.81, 150.21, 150.32, 153.03, 153.32, 158.78 (J_{CF}=244 Hz, J_{CF}=12 Hz), 162.59 (J_{CF}=248 Hz, J_{CF}=12 Hz), 169.07, 171.42.

### Example 4

### SN38-TBDPS-DGA-OH, Compound (7b)

10-OTBDPS-SN38 (compound SN38-TBDPS) is reacted with compound 6 in the same conditions as described in Example 3 to provide compound 7b.

### Example 5

### SCH-Glutaric-OH, Compound (9a)

Compound SCH AF (5.67 g, 8.10 mmol), DMAP (20.3 g, 166 mmol), and glutaric anhydride (compound 8, 18.9 g, 166 mmol) were dissolved in 600 mL anhydrous DCM and stirred overnight. The solution was then washed with 200 mL 0.1 N HCl three times and was evaporated to gun. It was then dissolved in 600 mL of acetonitrile/0.1 M sodium carbonate=1/1 solution and stirred for 4 h before the acetonitrile was evaporated. The product was extracted back to organic solvent DCM. The organic layer was dried over anhydrous MgSO₄. The solution was filtered and the solvent was removed in vacuo. The residue was dried under vacuum overnight to give the product (6.09 g, 7.47 mmol, 92%). ¹³C NMR δ 10.36, 17.27, 20.06, 22.30, 33.46, 37.43, 38.79, 49.06, 50.58, 53.37, 55.92, 60.23, 68.8670.69, 71.03, 83.93 (J_{CF}=4.7 Hz), 104.57 (J_{CF}=26 Hz), 111.27 (J_{CF}=24 Hz), 115.10, 116.59, 118.50, 123.50, 125.13, 125.48 (J_{CF}=12 Hz), 128.53 (J_{CF}=10 Hz, J_{CF}=5.4 Hz), 134.51, 144.53, 145.60, 150.55, 150.69, 153.03, 158.93 (J_{CF}=247 Hz, J_{CF}=12 Hz), 162.73 (J_{CF}=247 Hz, J_{CF}=12 Hz), 172.07.

### Example 6

### SN38-TBDPS-Glutaric-OH, Compound (9b)

Compound SN38-TBDPS is reacted with compound 8 in the same conditions as described in Example 5 to provide compound 8b.

### Example 7

### SCH-Succinic-OH, Compound (11a)

Compound SCH AF was reacted with succinic anhydride (compound 10) in the same conditions as described in Example 5 to provide compound 11a.

### Example 8

### SN38-TBDPS-Suceinic-OH, Compound (11b)

Compound SN38-TBDPS is reacted with compound 10 in the same conditions as described in Example 5 to provide compound 11b.

### Example 9

### PEG-[Lys (DGA-SCH AF)₂]₂, Compound (12a)

Compound 4 (0.5 g) was dissolved in 10 mL of anhydrous DCM and compound 7a (158 mg) and DMAP (71 mg) were added. The reaction mixture was cooled to 0° C. in an ice bath followed by addition of EDC (74 mg). The reaction mixture was stirred at room temperature overnight. The solvent was partially removed in vacuo and the residue was recrystallized three times from IPA, THF, and DCM-ether (4:11, v/v) in the order stated. The product was isolated and dried in the vacuum oven at 45° C. overnight to give the desired product (0.36 g, 64% yield). The amount of the SCH AF measured by US assay was 11% wt/wt: ¹³C NMR δ 9.71, 16.57, 21.41, 36.65, 68.09, 48.26, 49.75, 55.07, 59.52, 66.94, 67.01, 67.11, 67.14, 67.21, 67.29, 67.37, 67.42, 67.48, 67.76, 68.11, 68.83, 69.15, 70.71, 71.40, 71.54, 78.17, 78.30, 83.23, 103.84, 110.38, 110.67, 114.36, 115.68, 117.58, 122.72, 124.73, 124.84, 124.91, 127.86, 127.94, 134.33, 143.96, 144.97, 149.77, 149.82, 150.17, 152.18, 152.30, 152.43, 159.97, 160.15, 168.05, 168.21, 170.66.

### Example 10

### PEG-[Lys (DGA-SN38-TBDPS)₂]₂, Compound (12b)

Compound 7b is reacted with compound 4 in the same conditions as described in Example 9 to provide compound 12b.

### Example 11

### PEG-[Lys (Glutaric-SCH AF)₂]₂, Compound (13a)

Compound 8a is reacted with compound 4 in the same conditions as described in Example 9 to provide compound 13a.

### Example 12

### PEG-[Lys (Glutaric-SN38-TBDPS)₂]₂, Compound (13b)

Compound 8b is reacted with compound 4 in the same conditions as described in Example 9 to provide compound 13b.

### Example 13

### PEG-[Lys (Succinic-SCH AF)₂]₂, Compound (14a)

Compound 9a is reacted with compound 4 in the same conditions as described in Example 9 to provide compound 14a.

### Example 14

### PEG-[Lys (Succinic-SN38-TBDPS)₂]₂, Compound (14b)

Compound 9b is reacted with compound 4 in the same conditions as described in Example 9 to provide compound 14b.

### Example 15

### PEG-[Lys (DGA-SN38)₂]₂, Compound (15b)

A solution of TBAF (4 eq.) in a 1:1 mixture of THF and a 0.05 M HCl solution (v/v) was added to a solution of compound 12b in water The reaction mixture is stirred at room temperature for 4 hours and then, extracted with DCM twice. The combined organic layers are combined and dried over MgSO₄, filtered and evaporated under vacuum. The residue is dissolved in 7 volume equivalent of DMF and precipitated with 37 volume equivalent of IPA. The solid is filtered and washed with IPA. The precipitation with DMF/IPA is repeated Finally the residue is dissolved in DCM and precipitated by addition of ether. The solid is filtered and dried at 40° C. in vacuum oven overnight to provide the product.

### Example 16

### PEG-[Lys (Glutaric-SN38)₂]₂, Compound (16b)

Compound 13b is subjected to the same conditions as described in Example 15 to provide compound 16b.

### Example 17

### PEG-[Lys (Succinic-SN38)₂]₂, Compound (17b)

Compound 14b is subjected to the same conditions as described in Example 15 to provide compound 17b.

### Example 18

### PEG2-C3-amine, Compound (20)

PEG2-NHS (compound 18, Mw. 40 kDa, 0.0025 mmol) is dissolved in anhydrous DCM (10 mL) and 1,3-propyldiamine (0.01 mmol) is added to the solution. The reaction mixture was stirred at room temperature for overnight. The solvent is partially removed in vacuo and ethyl ether is added to precipitate the crude product, which is recrystallized from DCM-Ether to give the desired product.

### Example 19

### PEG2-[Lys (NHBoc)₂], Compound (21)

PEG2-amine (compound 20, 1.25 mmol) is azeotroped and toluene is removed in vacuo to dryness. The azeotroped PEG2-amine is dissolved in 200 mL of DCM and Boc-Lys-Boc (compound 2, 2.638 g, 5 mmol) and DMAP (610 mg, 5 mmol) are added and the reaction mixture is cooled to 0° C. for 15 minutes before the addition of EDC (958 mg, 5 mmol). The reaction mixture is allowed to warm to room temperature with stirring overnight. The solvent is removed in vacuo to dryness and the residue is recrystallized form IPA to give the product:

### Example 20

### PEG2-[Lys (NH₂)₂], Compound (22)

Compound 21 is dissolved in DCM (10 mL) and TEA (5 mL) is added slowly to die solution. The solution is stirred for 2 hours at the room temperature. The reaction solution is concentrated in vacuo and ethyl ether is added to precipitate the product. The product is isolated by filtration and dried overnight at 45° C. in vacuo.

### Example 21

### PEG2-[Lys (DGA-SCH AF)₂], Compound (23)

Compound 4 (0.5 g) is dissolved in 10 mL of anhydrous DCM and compound 7a (158 mg) and DIP (71 mg) are added. The reaction mixture is cooled to 0° C. in an ice bath followed by addition of EDC (74 mg). The reaction mixture is stirred at room temperature overnight. The solvent is partially removed in vacuo and the residue is recrystallized three times from IPA, THF, and DCM-ether (4:11, v/v) in the order stated. The product is isolated and dried in the vacuum oven at 45° C. overnight to give the product.

### Example 22

### Determination of Rates of Hydrolysis of PEG Prodrugs

The rates of hydrolysis were obtained by employing a C8 reversed phase column (Zorbax® SB-C8) using a gradient mobile phase made of (a) 0.1 M triethylammonium acetate buffer and (b) acetonitrile. A flow rate of 1 mL/min was used, and chromatograms were monitored using a UV detector. For hydrolysis in buffer, PEG derivatives were dissolved in 0.1 M pH 7.4 PBS at a concentration of 5 mg/mL, while for hydrolysis in plasma, the derivatives were dissolved in distilled water at a concentration of 20 mg/100 μL and 900 μL of rat plasma was added to this solution. The mixture was vortexed for 2 min and divided into 2 mL glass vials with 100 μL of the aliquot per each vial. The solutions were incubated at 37° C. for various periods of time. A mixture of methanol-acetonitrile (1:1, v/v, 400 μL) was added to a vial at the proper interval and the mixture was vortexed for 1 min, followed by filtration through 0.45 mm filter membrane (optionally followed by a second filtration through 0.2 mm filter membrane). An aliquot of 20 μL of the filtrate was injected into the HPLC. On the basis of the peak area, the amounts of native compound and PEG derivative were estimated, and the half-life of each compound in different media was calculated using linear regression analysis from the disappearance of PEG derivative. Compound 12a was subjected to hydrolysis and resulted in t_{1/2}=greater than 24 hours in pH 7.4 PBS buffer and t_{1/2}α=5 hours and t_{1/2}β=15 hours in rat plasma.

1. A compound of the Formula (I) 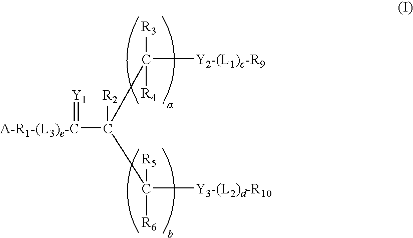
wherein:
R₁ is a substantially non-antigenic water-soluble polymer;
A is a capping group or 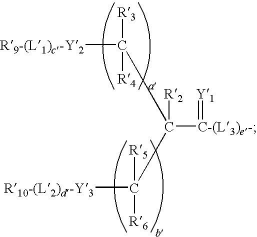
L₁₋₃ and L′₁₋₃ are independently selected bifunctional linkers;
Y₁ and Y′₁ are independently O, S, or NR₂₀;
Y₂₋₃ and Y′₂₋₃ are independently O, S, SO, SO₂ or NR₇;
R₂₋₇, R′₂₋₆, and R₂₀ are independently selected from the group consisting of hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, and substituted arylcarbonyloxy;
R₉₋₁₀ and R′₉₋₁₀ are independently selected from the group consisting of hydrogen, OH, leaving groups, functional groups, targeting groups, diagnostic agents and biologically active moieties;
(a) and (a′) are independently zero or a positive integer;
(b) and (b′) are independently a positive integer; and
(c), (c′), (d), (d′), (e) and (e′) are independently zero or 1.

2. The compound of claim 1, wherein the leaving group is selected from the group consisting of halogens, activated esters, imidazole, cyclic imide thione, N-hydroxysuccinimidyl, para-nitrophenoxy, N-hydroxyphtalimidyl, N-hydroxybenzotriazolyl, tosylate, mesylate, tresylate, nosylate, C₁-C₆ alkyloxy, C₁-C₆ alkanoyloxy, arylcarbonyloxy, ortho-nitrophenoxy, N-hydroxybenzotriazolyl, pentafluorophenoxy, 1,3,5-trichlorophenoxy, and 1,3,5-trifluorophenoxy.

3. The compound of claim 1 wherein the functional group is selected from the group consisting of maleimidyl, vinyl, residues of sulfone, amino, carboxy, mercapto, hydrazide, and carbazate.

4. The compound of claim 1, wherein R₉₋₁₀ and R′₉₋₁₀ are independently selected from the group consisting of OH, methoxy, tert-butoxy, para-nitrophenoxy and N-hydroxysuccinimidyl.

5. The compound of claim 1 wherein the biologically active moiety is selected from the group consisting of —NH₂ containing moieties, —OH containing moieties and —SH containing moieties.

6. The compound of claim 1, wherein the biologically active moiety is selected from the group consisting of pharmaceutically active compounds, enzymes, proteins, oligonucleotides, antibodies, monoclonal antibodies, single chain antibodies and peptides.

7. The compound of claim 1, wherein L₁₋₃ and L′₁₋₃ are independently selected from the group consisting of:
—[C(═O)]ᵥ(CR₂₂R₂₃)ₜ[C(═O)]_{v′}—,
—[C(═O)]ᵥ(CR₂₂R₂₃)ₜ—O[C(═O)]_{v′}—,
—[C(═O)]ᵥ(CR₂₂R₂₃)ₜ—NR₂₆[C(═O)]_{v′}—,
—[C(═O)]ᵥO(CR₂₂R₂₃)ₜ[C(═O)]_{v′}—,
—[C(═O)]ᵥO(CR₂₂R₂₃)ₜO[C(═O)]_{v′}—,
—[C(═O)]ᵥO(CR₂₂R₂₃)ₜNR₂₆[C(═O)]_{v′}—,
—[C(═O)]ᵥNR₂₁(CR₂₂R₂₃)ₜ[C(═O)]_{v′}—,
—[C(═O)]ᵥNR₂₁(CR₂₂R₂₃)ₜO[C(═O)]_{v′}—,
—[C(═O)]ᵥNR₂₁(CR₂₂R₂₃)ₜNR₂₆[C(═O)]_{v′}—,
—[C(═O)]ᵥ(CR₂₂R₂₃)ₜO—(CR₂₈R₂₉)_{t′}[C(═O)]_{v′}—,
—[C(═O)]ᵥ(CR₂₂R₂₃)ₜNR₂₆—(CR₂₈R₂₉)_{t′}[C(═O)]_{v′}—,
—[C(═O)]ᵥ(CR₂₂R₂₃)ₜS—(CR₂₈R₂₉)_{t′}[C(═O)]_{v′}—,
—[C(═O)]ᵥO(CR₂₂R₂₃)ₜO—(CR₂₈R₂₉)_{t′}[C(═O)]_{v′}—,
—[C(═O)]ᵥO(CR₂₂R₂₃)ₜNR₂₆—(CR₂₈R₂₉)_{t′}[C(═O)]_{v′}—,
—[C(═O)]ᵥO(CR₂₂R₂₃)ₜS—(CR₂₈R₂₉)_{t′}[C(═O)]_{v′}—,
—[C(═O)]ᵥNR₂₁(CR₂₂R₂₃)ₜO—(CR₂₈R₂₉)_{t′}[C(═O)]_{v′}—,
—[C(═O)]ᵥNR₂₁(CR₂₂R₂₃)ₜNR₂₆—(CR₂₈R₂₉)_{t′}[C(═O)]_{v′}—,
—[C(═O)]ᵥNR₂₁(CR₂₂R₂₃)ₜS—(CR₂₈R₂₉)_{t′}[C(═O)]_{v′}—,
—[C(═O)]ᵥ(CR₂₂R₂₃CR₂₈R₂₉O)ₜNR₂₆[C(═O)]_{v′}—,
—[C(═O)]ᵥ(CR₂₂R₂₃CR₂₈R₂₉O)ₜ[C(═O)]_{v′}—,
—[C(═O)]ᵥO(CR₂₂R₂₃CR₂₈R₂₉O)ₜNR₂₆[C(═O)]_{v′}—,
—[C(═O)]ᵥO(CR₂₂R₂₃CR₂₈R₂₉O)ₜ[C(═O)]_{v′}—,
—[C(═O)]ᵥNR₂₁(CR₂₂R₂₃CR₂₈R₂₉O)ₜNR₂₆[C(═O)]_{v′}—,
—[C(═O)]ᵥNR₂₁(CR₂₂R₂₃CR₂₈R₂₉O)ₜ[C(═O)]_{v′}—,
—[C(═O)]ᵥ(CR₂₂R₂₃CR₂₈R₂₉O)ₜ(CR₂₄R₂₅)_{t′}[C(═O)]_{v′}—,
—[C(═O)]ᵥO(CR₂₂R₂₃CR₂₈R₂₉O)ₜ(CR₂₄R₂₅)_{t′}[C(═O)]_{v′}—,
—[C(═O)]ᵥNR₂₁(CR₂₂R₂₃CR₂₈R₂₉O)ₜ(CR₂₄R₂₅)_{t′}[C(═O)]_{v′}—,
—[C(═O)]ᵥ(CR₂₂R₂₃CR₂₈R₂₉O)ₜ(CR₂₄R₂₅)_{t′}O[C(═O)]_{v′}—,
—[C(═O)]ᵥ(CR₂₂R₂₃)ₜ(CR₂₄R₂₅CR₂₈R₂₉O)_{t′}[C(═O)]_{v′}—,
—[C(═O)]ᵥ(CR₂₂R₂₃)ₜ(CR₂₄R₂₅CR₂₈R₂₉O)_{t′}NR₂₆[C(═O)]_{v′}—,
—[C(═O)]ᵥO(CR₂₂R₂₃CR₂₈R₂₉O)ₜ(CR₂₄R₂₅)_{t′}O[C(═O)]_{v′}—,
—[C(═O)]ᵥO(CR₂₂R₂₃)ₜ(CR₂₄R₂₅CR₂₈R₂₉O)_{t′}[C(═O)]_{v′}—,
—[C(═O)]ᵥO(CR₂₂R₂₃)ₜ(CR₂₄CR₂₅CR₂₈R₂₉O)_{t′}NR₂₆[C(═O)]_{v′}—,
—[C(═O)]ᵥNR₂₁(CR₂₂R₂₃CR₂₈R₂₉O)ₜ(CR₂₄R₂₅)_{t′}O[C(═O)]_{v′}—,
—[C(═O)]ᵥNR₂₁(CR₂₂R₂₃)ₜ(CR₂₄R₂₅CR₂₈R₂₉O)_{t′}[C(═O)]_{v′}—,
—[C(═O)]ᵥNR₂₁(CR₂₂R₂₃)ₜ(CR₂₄R₂₅CR₂₈R₂₉O)_{t′}NR₂₆[C(═O)]_{v′}—, 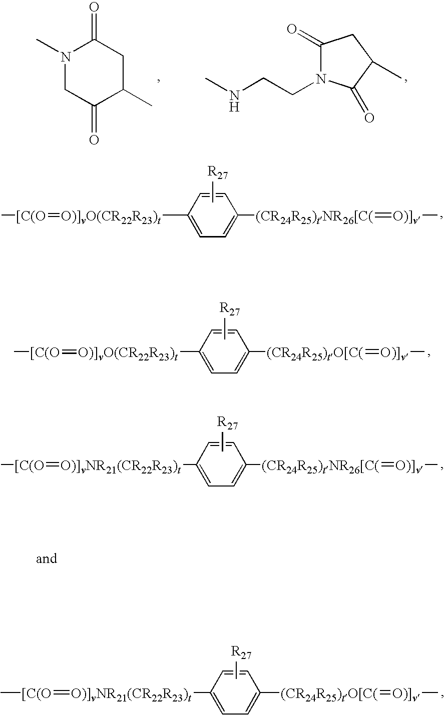
wherein:
R₂₁₋₂₉ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
(t) and (t′) are independently zero or a positive integer; and
(v) and (v′) are independently zero or 1.

8. The compound of claim 1, wherein L₁₋₃ and L′₁₋₃ are independently selected from the group consisting of: 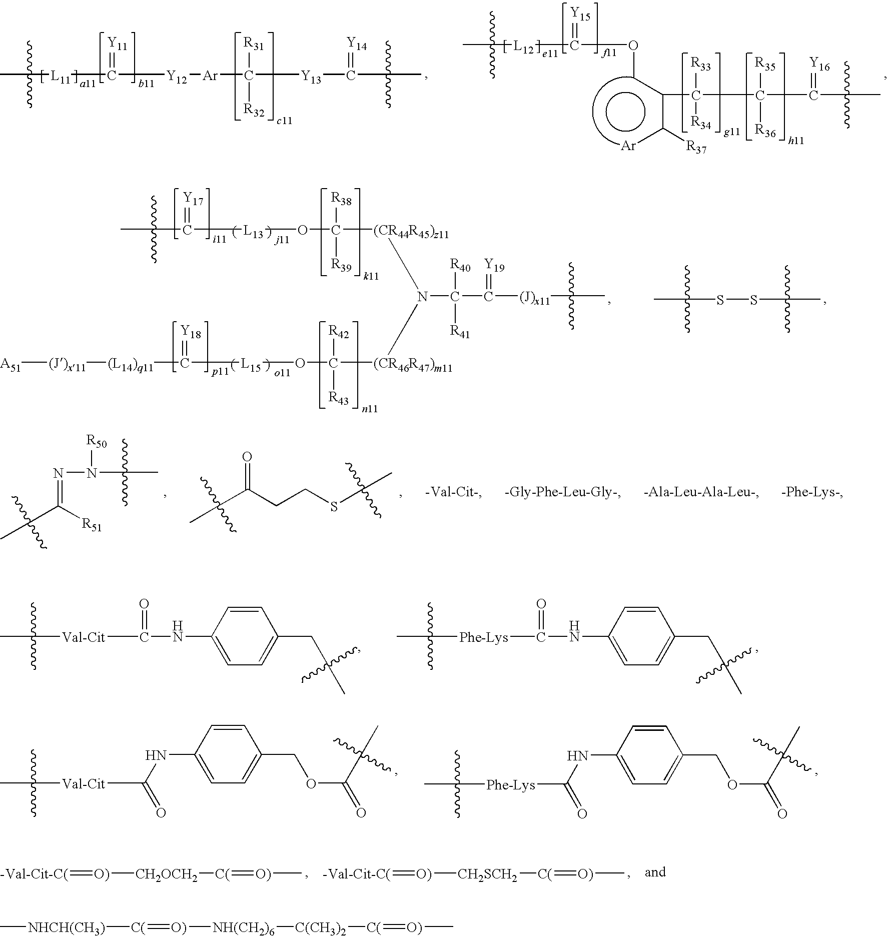
wherein,
Y₁₁₋₁₉ are independently O, S or NR₄₈;
R₃₁₋₄₈, R₅₀₋₅₁ and A₅₁ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
Ar is an aryl or heteroaryl moiety;
L₁₁₋₁₅ are independently selected bifunctional spacers;
J and J′ are independently selected from selected from the group consisting of moieties actively transported into a target cell, hydrophobic moieties, bifunctional linking moieties and combinations thereof;
(c11), (h11), (k11), (z11), (m11) and (n11) are independently selected positive integers;
(a11), (e11), (g11), (j11), (o11) and (q11) are independently either zero or a positive integers; and
(b11), (x11), (x′11), (f11), (i11) and (p11) are independently zero or one.

9. The compound of claim 1, wherein L₁₋₃ and L′₁₋₃ are independently selected from the group consisting of:
—[C(═O)]ᵣNH(CH₂)₂CH═N—NHC(═O)—(CH₂)₂—,
—[C(═O)]ᵣNH(CH₂)₂(CH₂CH₂O)₂(CH₂)₂NH[C(═O)]_{r′}—,
—[C(═O)]ᵣNH(CH₂CH₂)(CH₂CH₂O)₂NH[C(═O)]_{r′}—,
—[C(═O)]ᵣNH(CH₂CH₂)ₛNH(CH₂CH₂)_{s′}[C(═O)]_{r′}—,
—[C(═O)]ᵣNH(CH₂CH₂)ₛS(CH₂CH₂)_{s′}[C(═O)]_{r′}—,
—[C(═O)]ᵣNH(CH₂CH₂)(CH₂CH₂O)[C(═O)]_{r′}—,
—[C(═O)]ᵣNH(CH₂CH₂)ₛO(CH₂CH₂)_{s′}[C(═O)]_{r′}—,
—[C(═O)]ᵣNH(CH₂CH₂O)(CH₂)NH[C(═O)]_{r′}—,
—[C(═O)]ᵣNH(CH₂CH₂O)₂(CH₂)[C(═O)]_{r′}—,
—[C(═O)]ᵣNH(CH₂CH₂O)ₛ(CH₂)_{s′}[C(═O)]_{r′}—,
—[C(═O)]ᵣNHCH₂CH₂NH[C(═O)]_{r′}—,
—[C(═O)]ᵣNH(CH₂CH₂)₂O[C(═O)]_{r′}—,
—[C(═O)]ᵣNH(CH₂CH₂O)[C(═O)]_{r′}—,
—[C(═O)]ᵣNH(CH₂CH₂O)₂[C(═O)]_{r′}—,
—[C(═O)]ᵣNH(CH₂)₃[C(═O)]_{r′}—,
—[C(═O)]ᵣO(CH₂CH₂O)₂(CH₂)[C(═O)]_{r′}—,
—[C(═O)]ᵣO(CH₂)₂NH(CH₂)₂[C(═O)]_{r′}—,
—[C(═O)]ᵣO(CH₂CH₂O)₂NH[C(═O)]_{r′}—,
—[C(═O)]ᵣO(CH₂)₂O(CH₂)₂[C(═O)]_{r′}—,
—[C(═O)]ᵣO(CH₂)₂S(CH₂)₂[C(═O)]_{r′}—,
—[C(═O)]ᵣO(CH₂CH₂)NH[C(═O)]_{r′}—,
—[C(═O)]ᵣO(CH₂CH₂)O[C(═O)]_{r′}—,
—[C(═O)]ᵣO(CH₂)₃NH[C(═O)]_{r′}—,
—[C(═O)]ᵣO(CH₂)₃O[C(═O)]_{r′}—,
—[C(═O)]ᵣO(CH₂)₃[C(═O)]_{r′}—,
—[C(═O)]ᵣCH₂NHCH₂[C(═O)]_{r′}—,
—[C(═O)]ᵣCH₂OCH₂[C(═O)]_{r′}—,
—[C(═O)]ᵣCH₂SCH₂[C(═O)]_{r′}—,
—[C(═O)]ᵣS(CH₂)₃[C(═O)]_{r′}—,
—[C(═O)]ᵣ(CH₂)₃[C(═O)]_{r′}—, 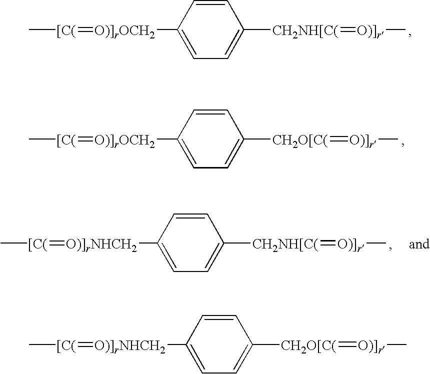
wherein, (r) and (r′) are independently zero or 1.

10. The compound of claim 1, wherein L₁₋₃ and L′₁₋₃ are independently selected from the group consisting of amino acids, amino acid derivatives, and peptides.

11. The compound of claim 1 having the formula (II)

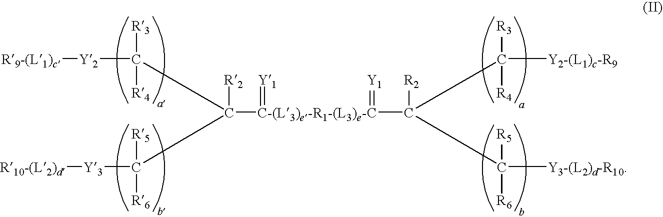

12. The compound of claim 1, wherein A is selected from the group consisting of H, NH₂, OH, CO₂H, C₁₋₆ alkoxy and C₁₋₆ alkyl.

13. The compound of claim 1, wherein R₁ comprises a linear, terminally branched or multi-armed polyalkylene oxide.

14. The compound of claim 13, wherein the polyalkylene oxide is selected from the group consisting of polyethylene glycol and polypropylene glycol.

15. The compound of claim 13, wherein the polyalkylene oxide is selected from the group consisting of;
—Y₇₁—(CH₂CH₂O)ₙ—CH₂CH₂—Y₇₁—,
—Y₇₁-(CH₂CH₂O)ₙ—CH₂C(═Y₇₂)—Y₇₁—,
—Y₇₁—C(═Y₇₂)—(CH₂)ₐ₇₁—Y₇₃—(CH₂CH₂O)ₙ—CH₂CH₂—Y₇₃—(CH₂)ₐ₇₁—C(═Y₇₂)—Y₇₁—, and
—Y₇₁—(CR₇₁R₇₂)ₐ₇₂—Y₇₃—(CH₂)_{b71}—O—(CH₂CH₂O)ₙ—(CH₂)_{b71}—Y₇₃—(CR₇₁R₇₂)ₐ₇₂—Y₇₁—,
wherein:
Y₇₁ and Y₇₃ are independently O, S, SO, SO₂, NR₇₃ or a bond;
Y₇₂ is O, S, or NR₇₄;
R₇₁, R₇₂, R₇₃, and R₇₄ are independently selected from the same moieties which can be used for R₂;
(a71), (a72), and (b71) are independently zero or positive integers; and
(n) is an integer from about 10 to about 2300.

16. The compound of claim 13, wherein the polyalkylene oxide is a polyethylene glycol of the formula, —O—(CH₂CH₂O)ₙ—
wherein (n) is an integer from about 10 to about 2,300.

17. The compound of claim 1, wherein R₁ has an average molecular weight from about 2,000 to about 100,000 daltons.

18. The compound of claim 1, wherein R₁ has an average molecular weight of from about 5,000 to about 60,000 daltons.

19. The compound of claim 1, wherein R₁ has an average molecular weight from about 5,000 to about 25,000 daltons or from about 20,000 to about 45,000 daltons.

20. The compound of claim 1 wherein R₂₋₈ and R′₂₋₈ are independently selected from the group consisting of hydrogen, methyl, ethyl and isopropyl.

21. A compound of claim 1 selected from the group consisting of: 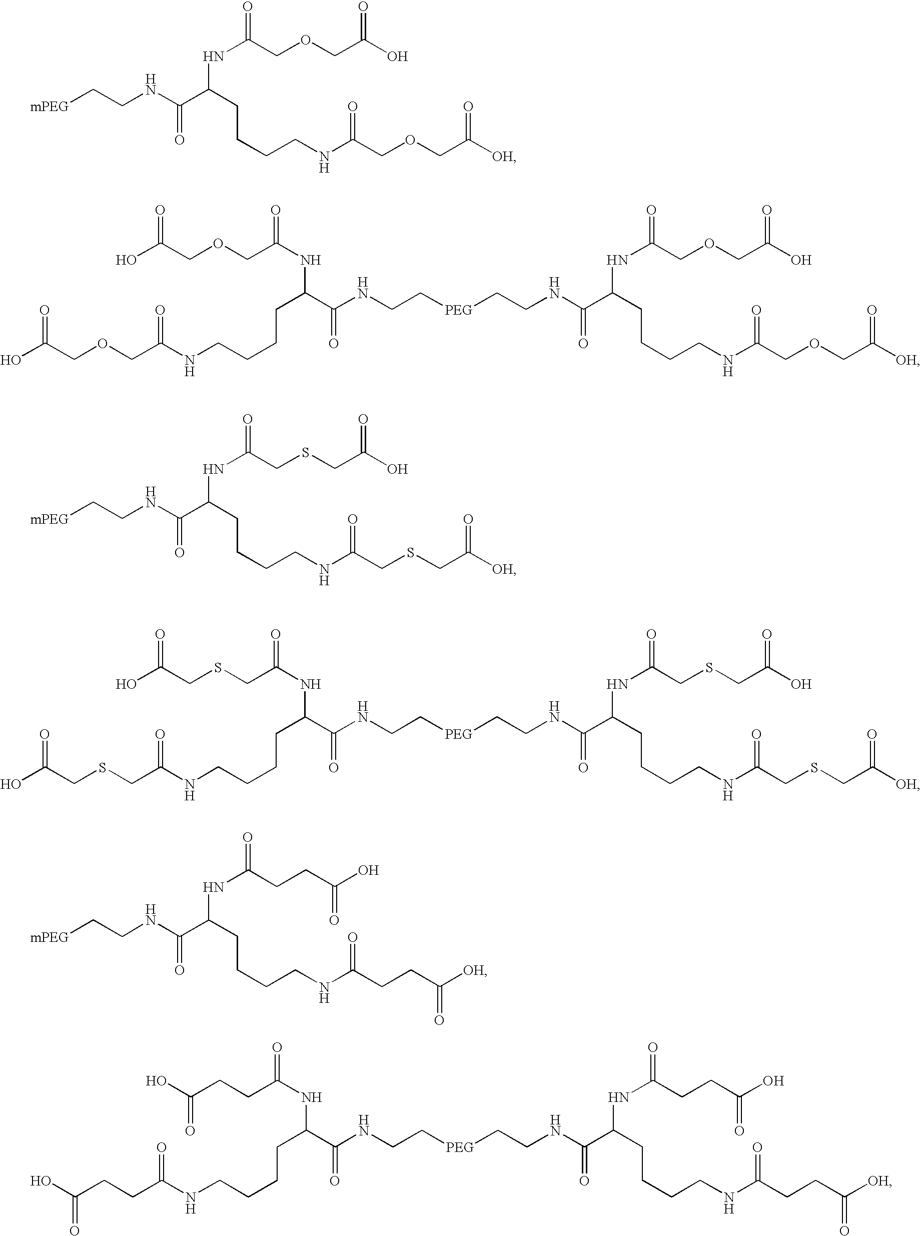 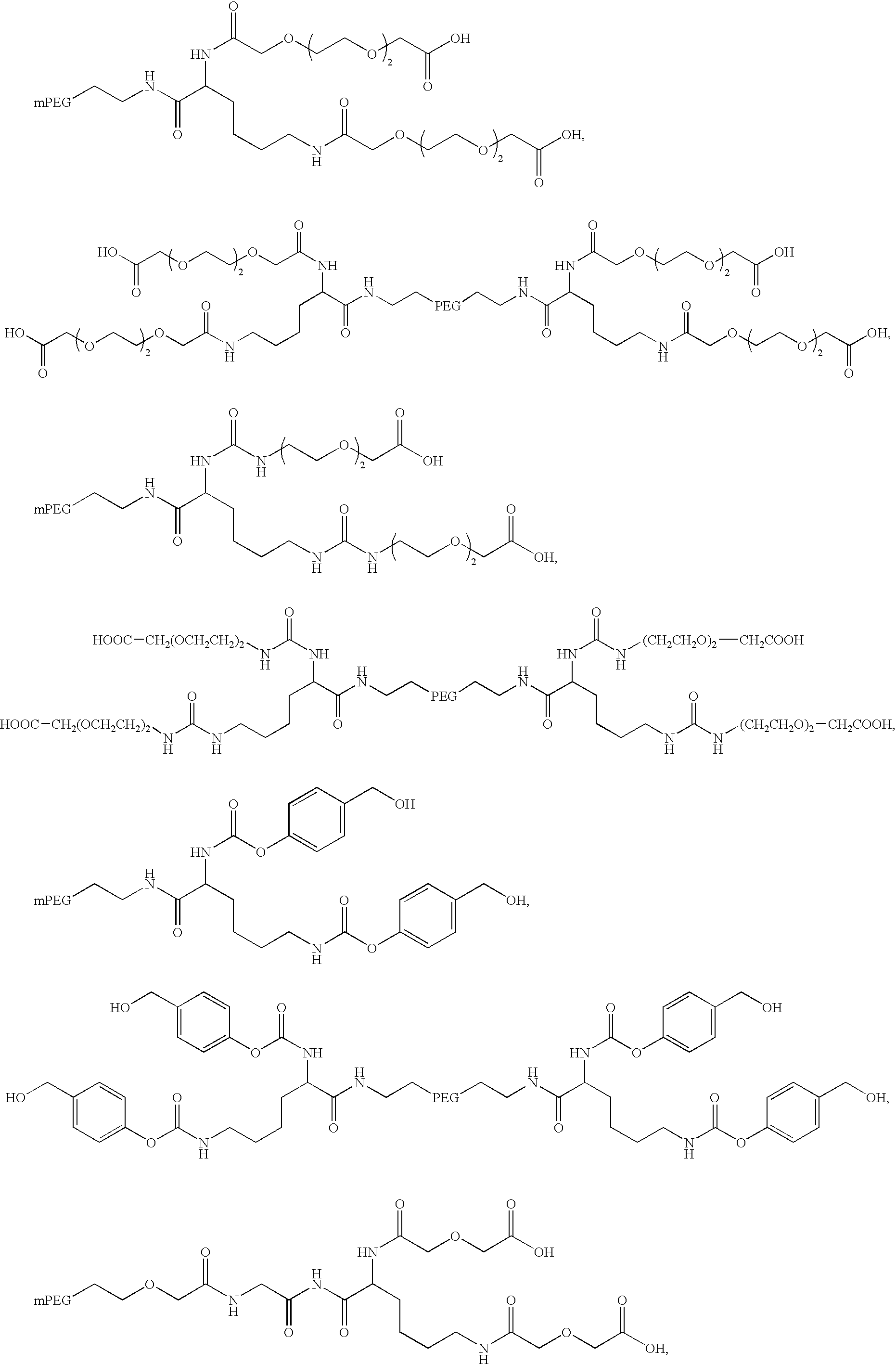 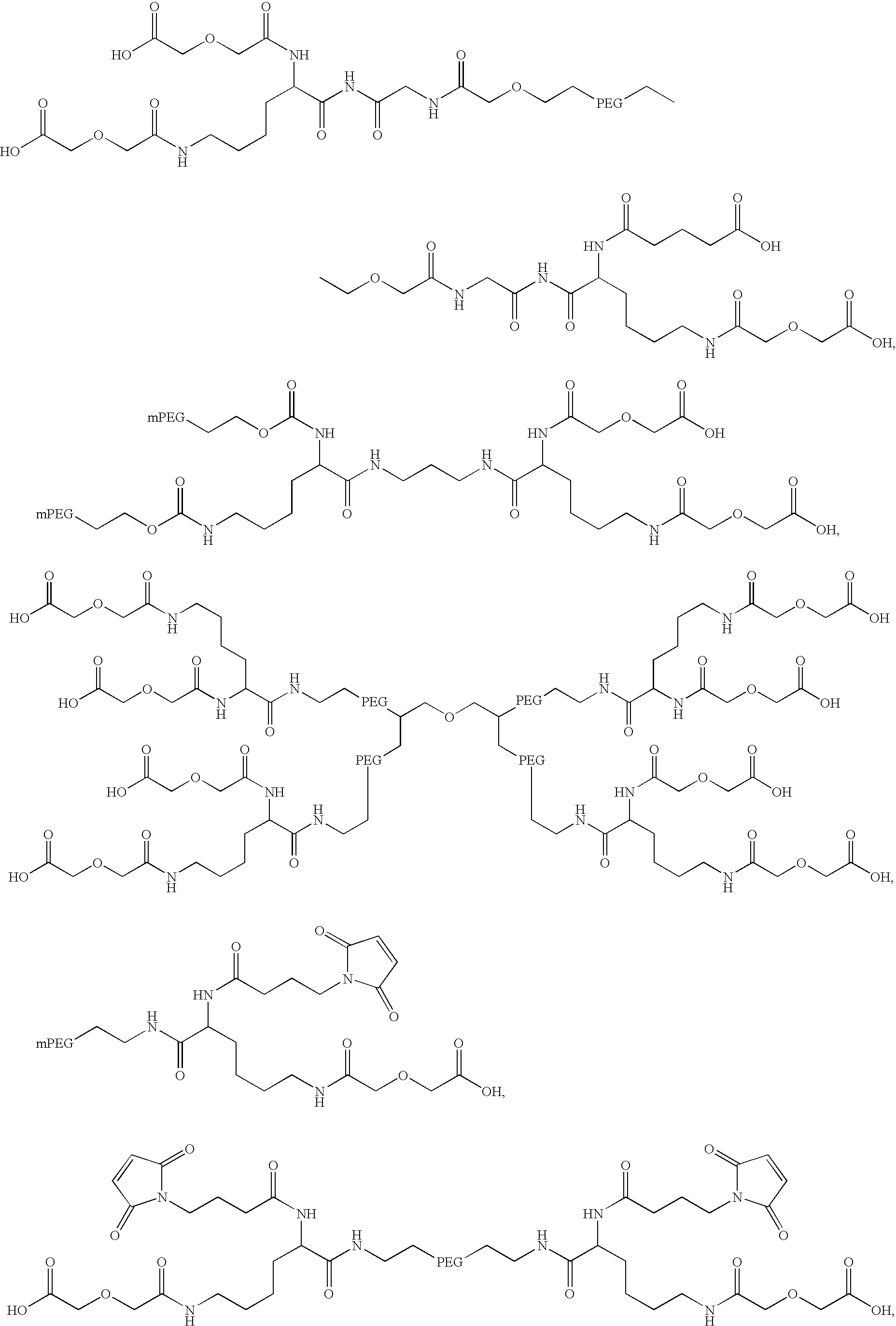 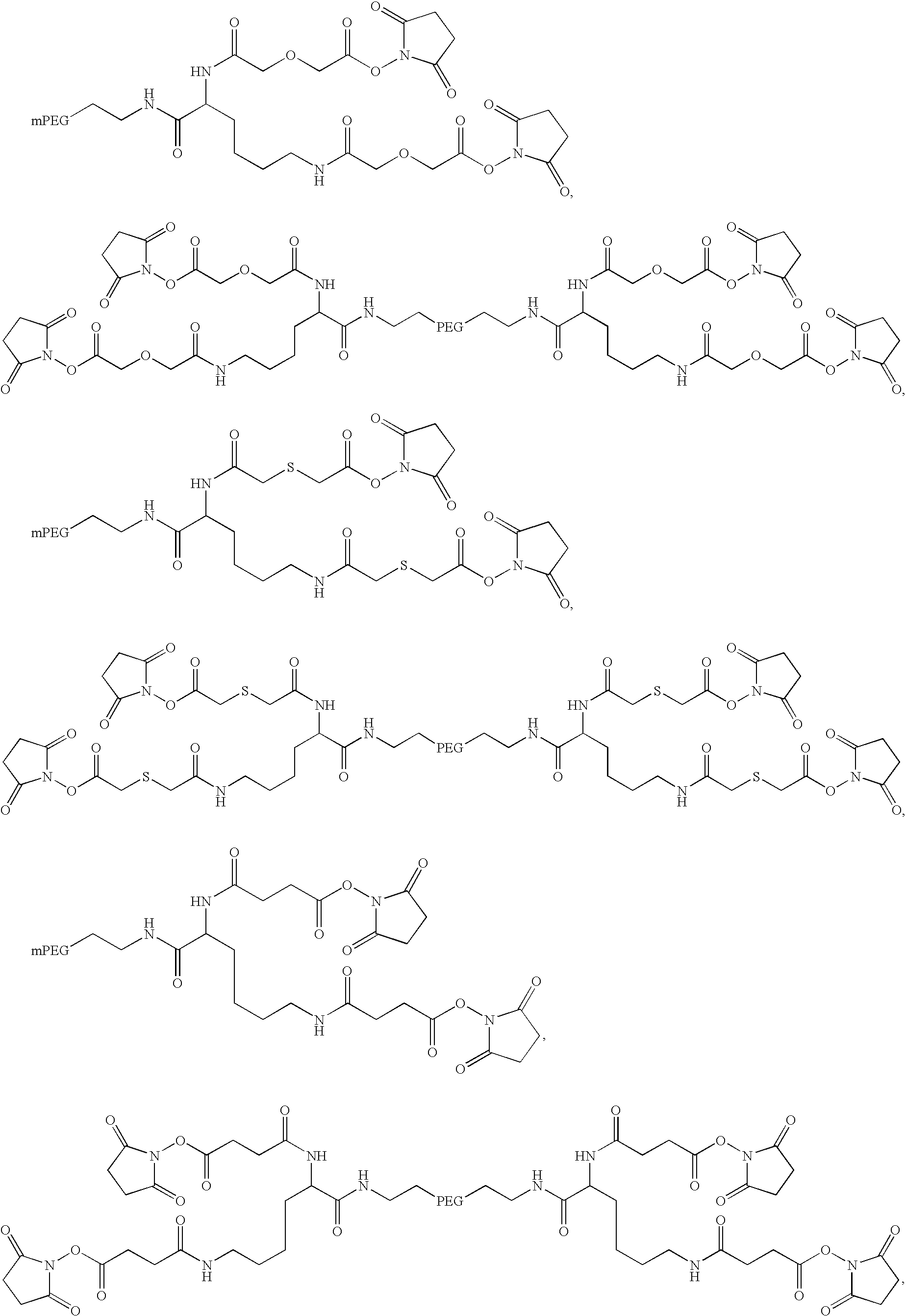 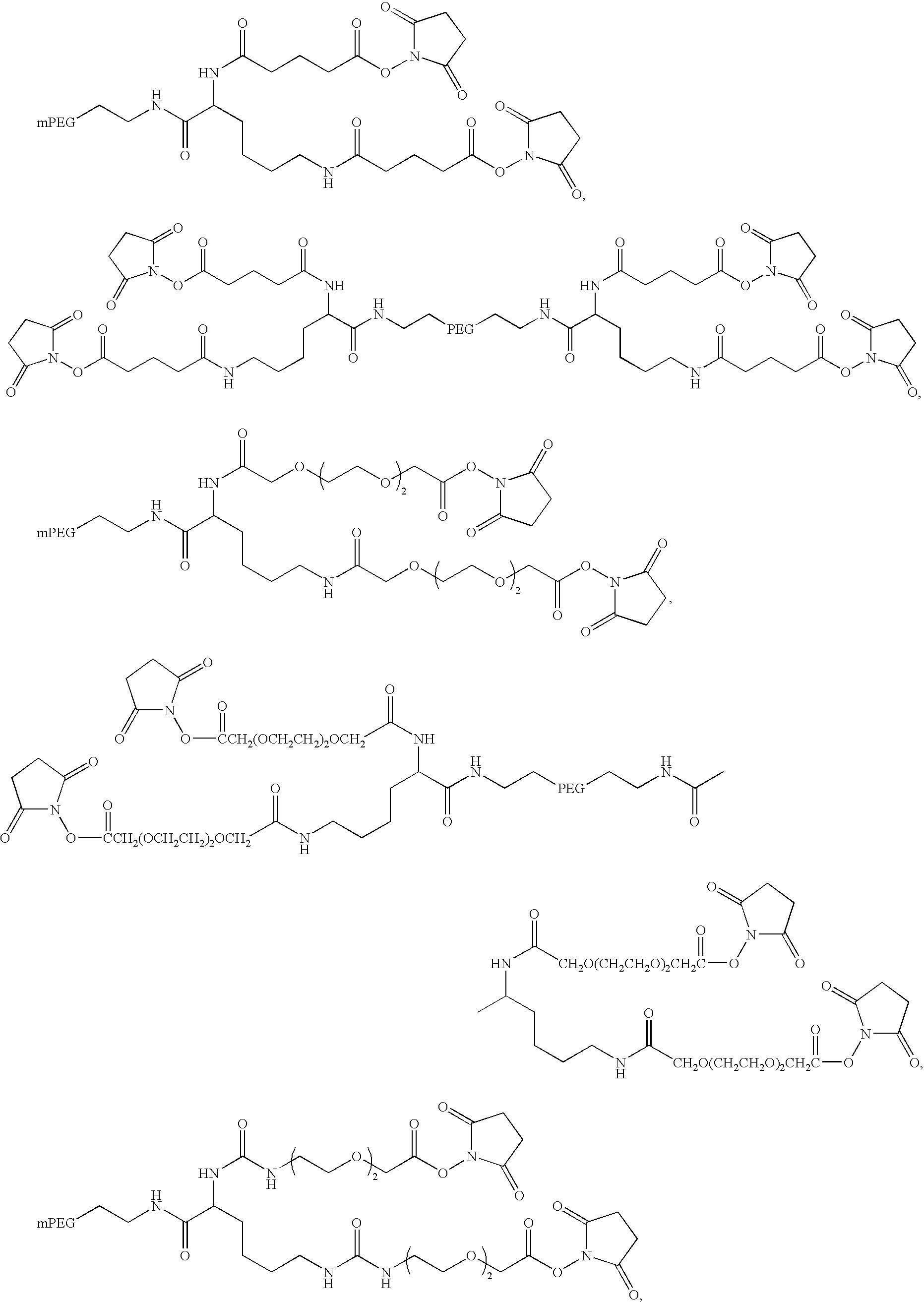 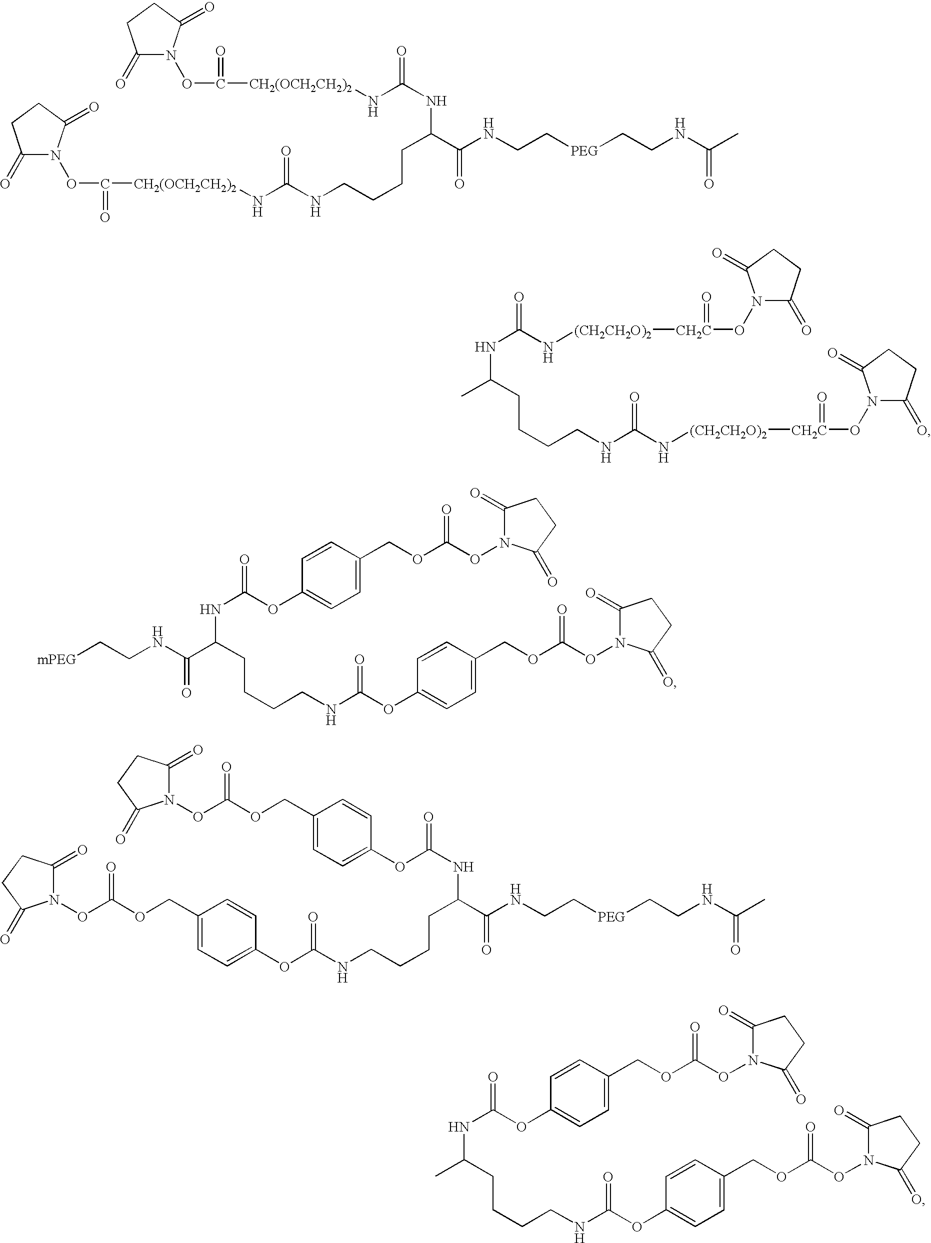 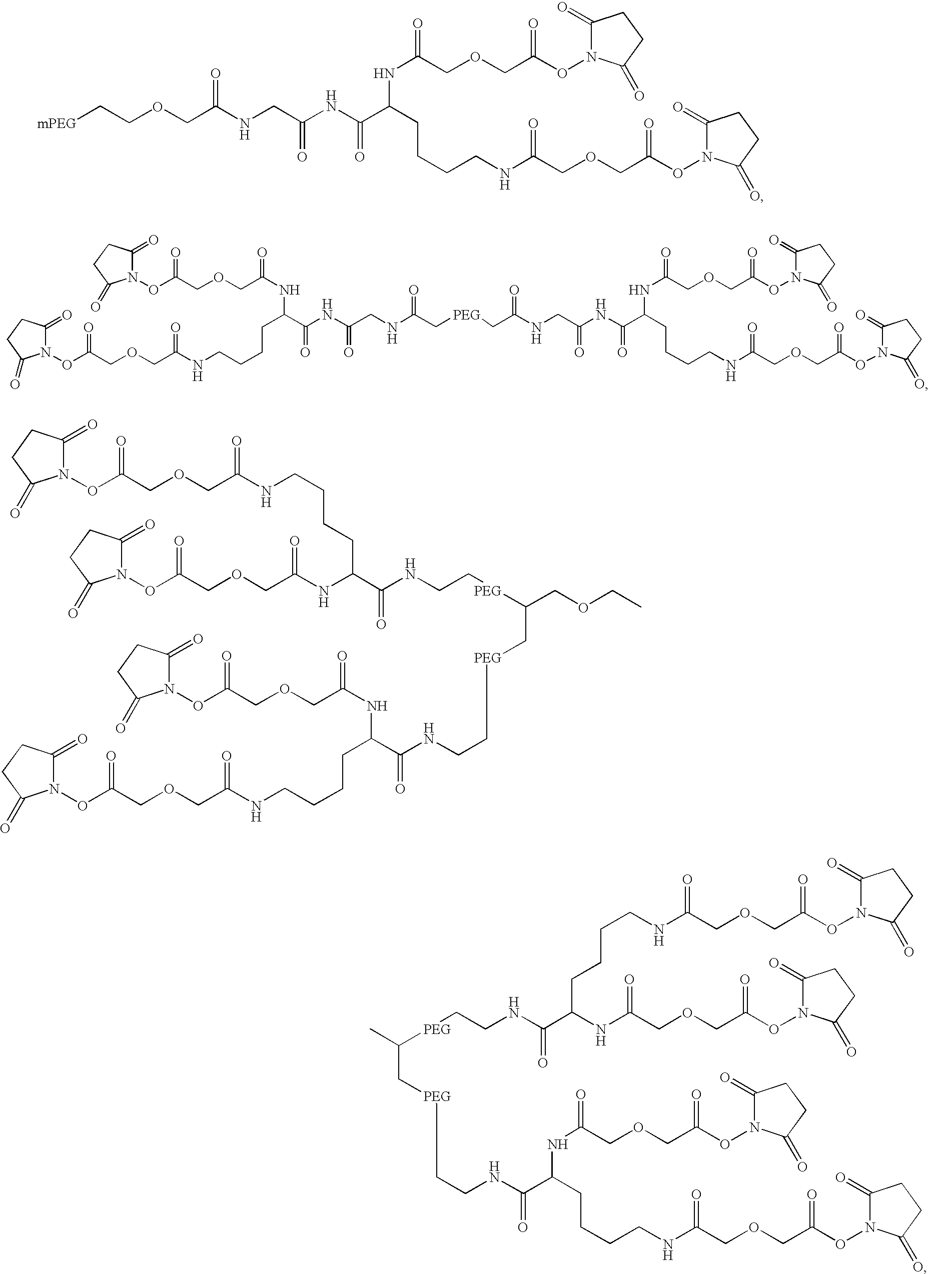 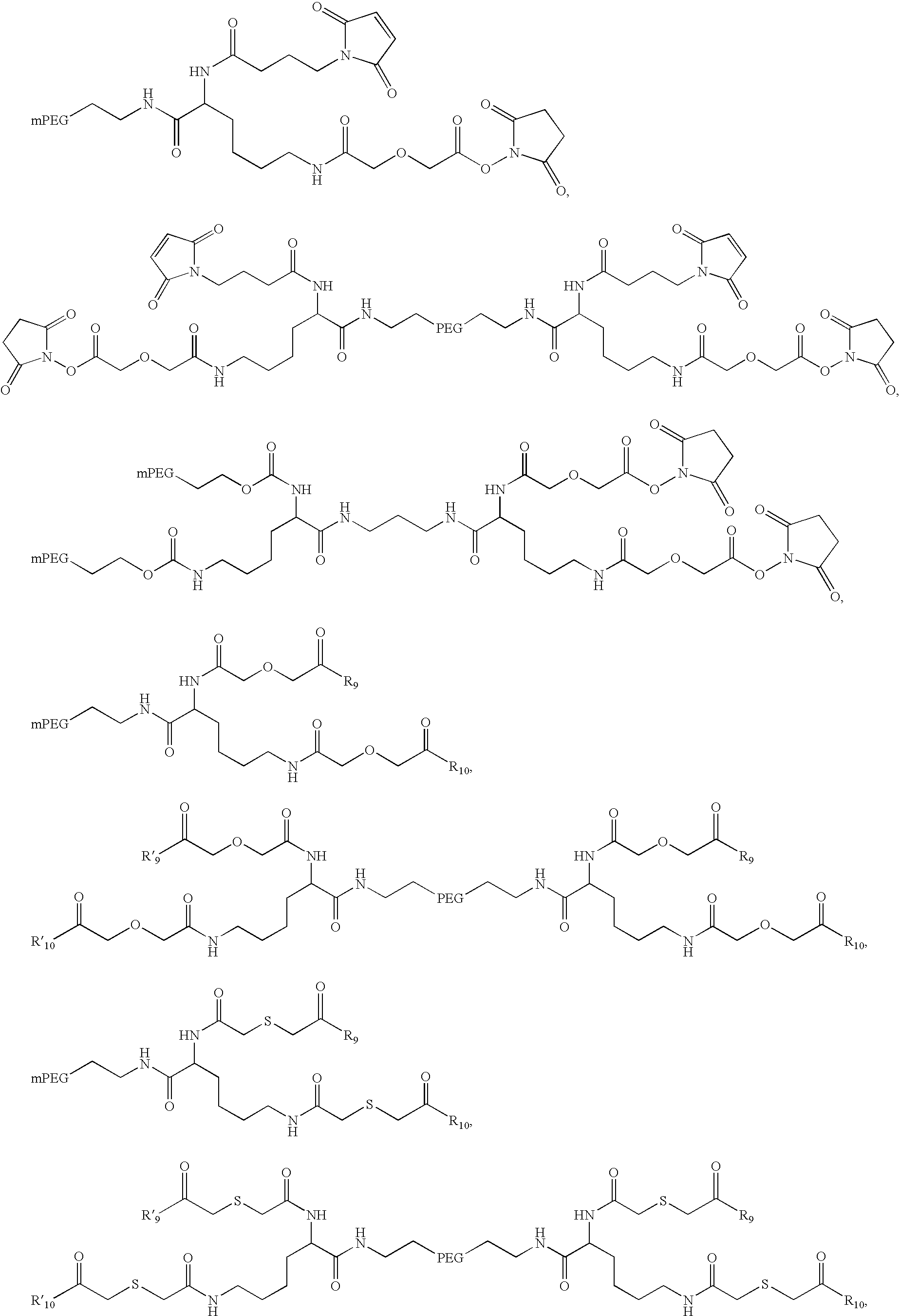 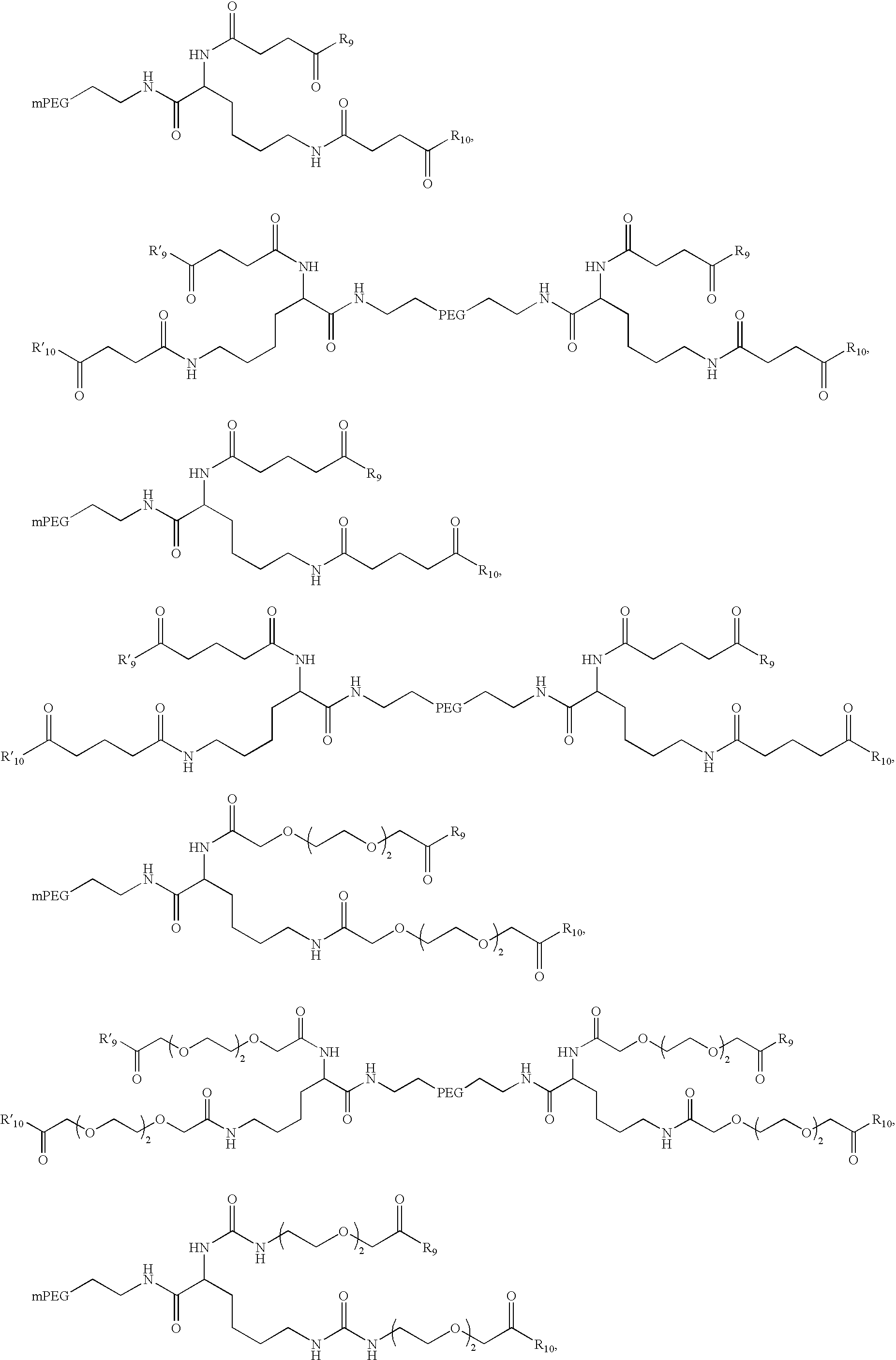 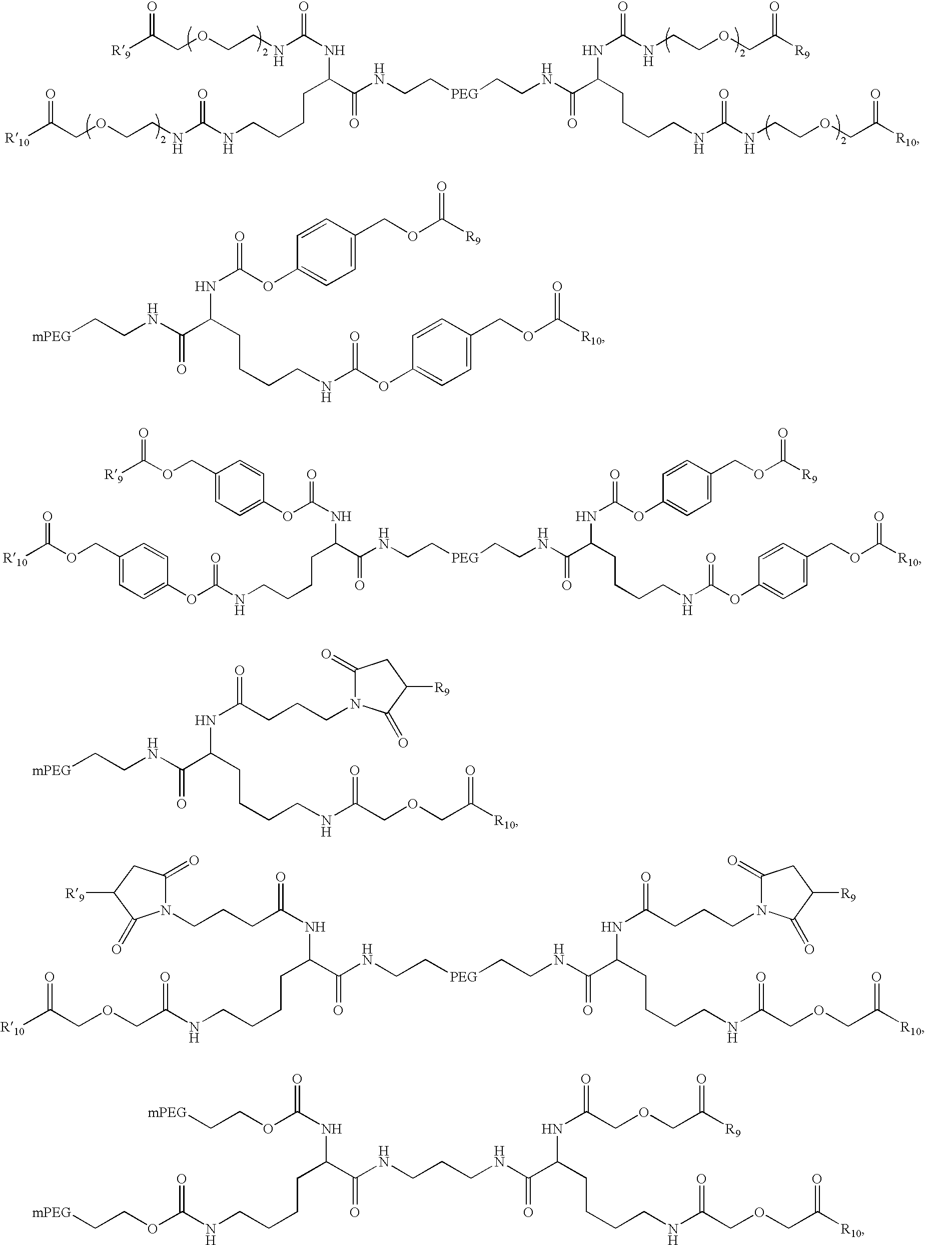 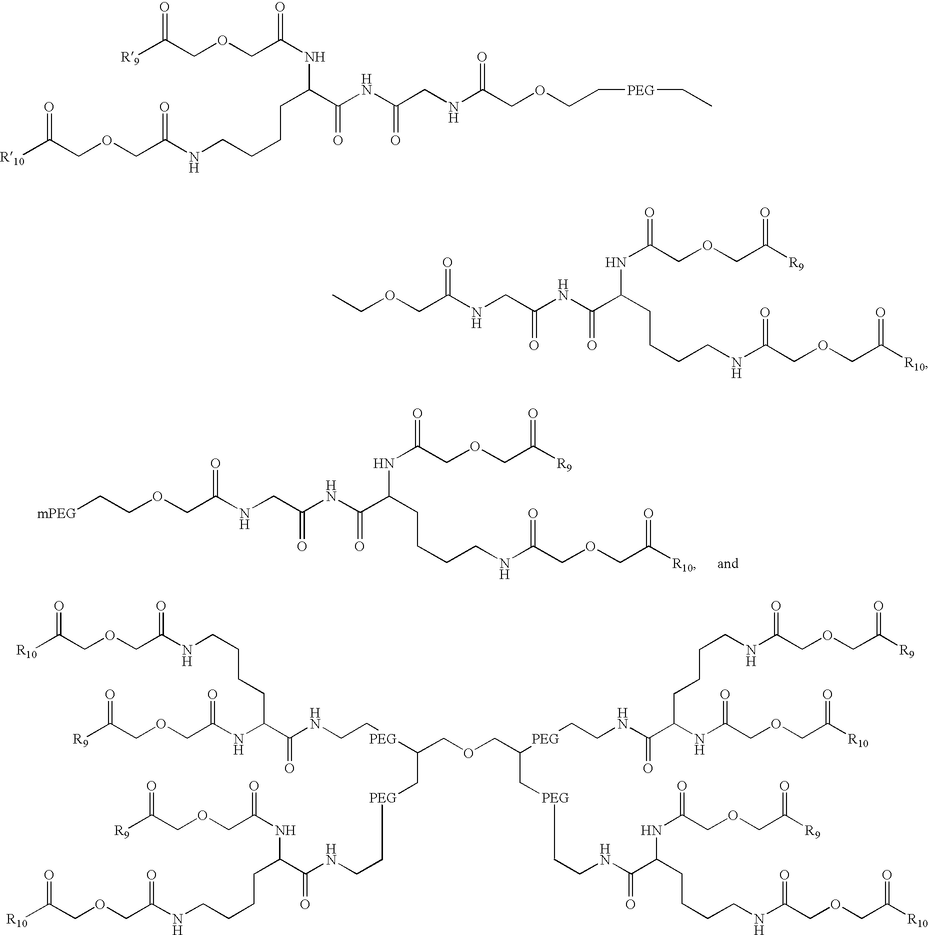
wherein:
mPEG has the formula CH₃O(CH₂CH₂O)ₙ—;
PEG has the formula —O(CH₂CH₂O)ₙ—,
(n) is an integer from about 10 to about 2,300; and
R₉₋₁₀ and R′₉₋₁₀ are independently selected from the group consisting of targeting groups, diagnostic agents and biologically active moieties.

22. A compound of claim 1 selected from the group consisting of: 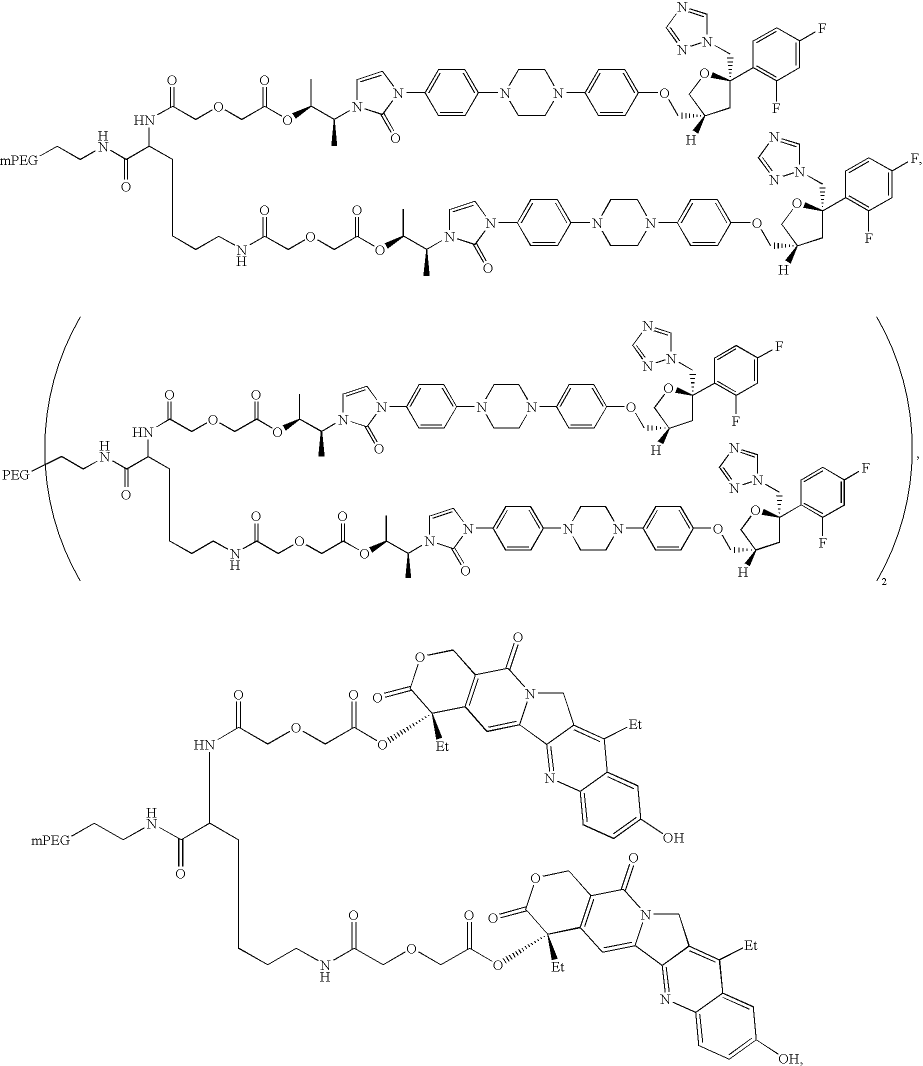 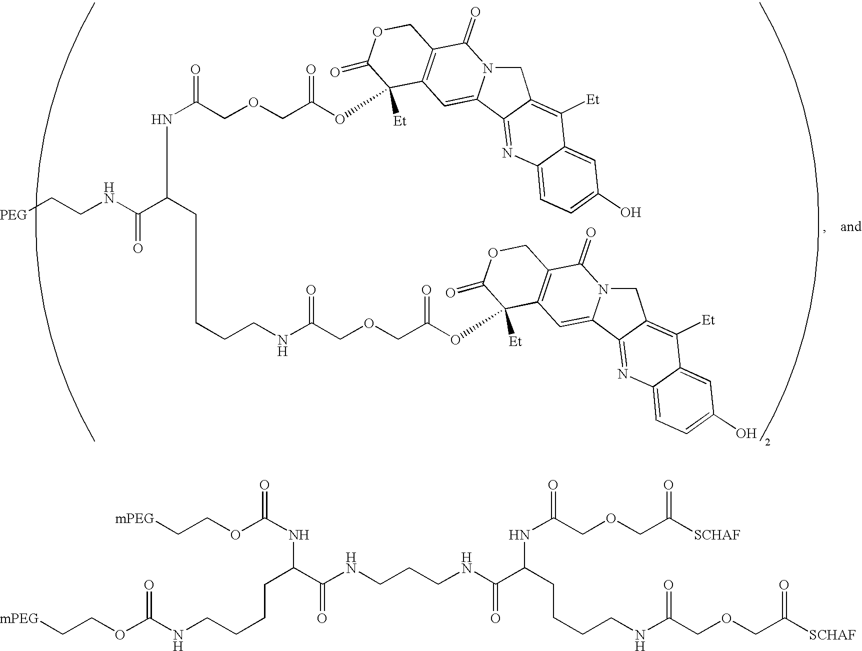
wherein:
SCH AF is 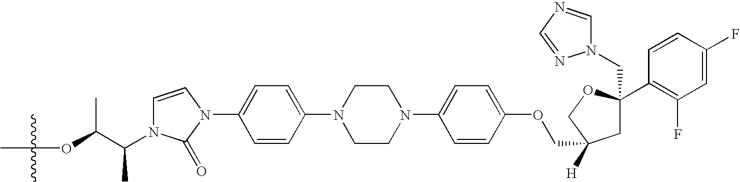
mPEG has the formula CH₃—O(CH₂CH₂O)ₙ—;
PEG has the formula —O(CH₂CH₂O)ₙ—, and
(n) is an integer from about 10 to about 2,300.

23. A method of preparing a polymeric conjugate having a branching moiety comprising:
(i) reacting a compound of Formula (III):
A₁-R₁-M₁ (III)
with a compound of Formula (VI) 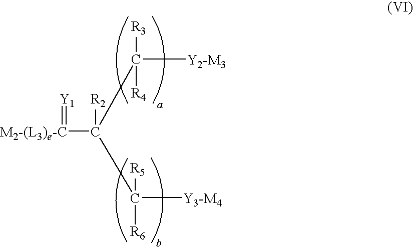
under conditions sufficient to form a compound of Formula (V): 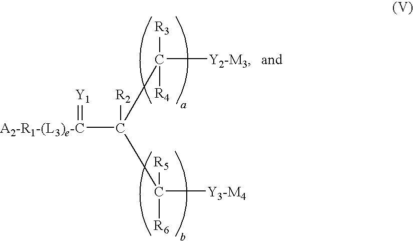
(ii) deprotecting the compound of Formula (V), under sufficient conditions to form a compound of Formula (V′): 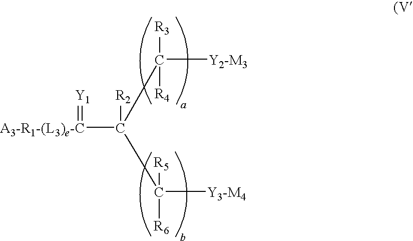
wherein:
R₁ is a substantially non-antigenic water-soluble polymer;
A₁ is a capping group or M₁;
A₂ is a capping group or 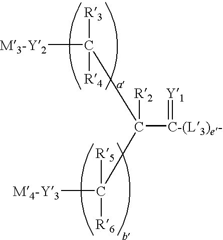
A₃ is a capping group or 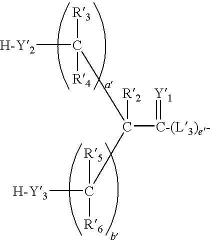
M₁ is —OH, SH, or —NHR₃₀;
M₂ is OH or a leaving group;
M₃₋₄ and M′₃₋₄ are independently selected protecting groups;
L₃ and L′₃ are independently selected bifunctional linkers;
Y₁ and Y′₁ are independently O, S, or NR₂₀;
Y₂₋₃ and Y′₂₋₃ are independently O, S, SO, SO₂ or NR₇;
R₂₋₇, R′₂₋₆, R₂₀ and R₃₀ are independently selected from the group consisting of hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy;
(a) and (a′) are independently zero or a positive integer;
(b) and (b′) are independently a positive integer; and
(e) and (e′) are independently zero or 1.

24. The method of claim 23 further comprising:
reacting the deprotected compound of Formula (V′) with a compound of Formula (VI):
M₅-(L″₁)_{c}-R″₉ (VI)
under conditions sufficient to form a compound of Formula (VII) 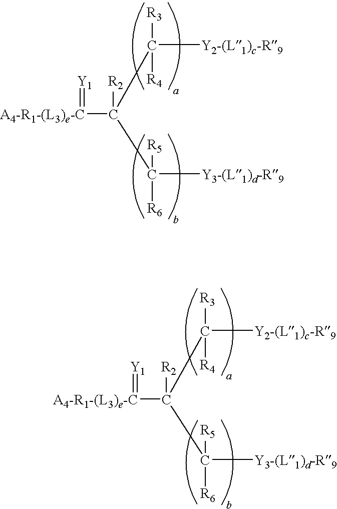
wherein
each R″₉ is independently a targeting group, a diagnostic agent or a biologically active moiety;
A₄ is a capping group or 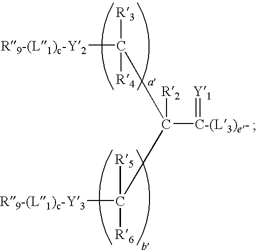
M₅ is —OH or a leaving group;
each L″₁ is independently a bifunctional linker;
each (c) is independently zero or 1; and
all other variables are as defined in claim 23.

25. A method of treating a mammal comprising administering an effective amount of a compound of Formula (I) to a patient in need thereof.
